(19) United States
(12) Patent Application Publication
(10) Pub. No.: US 2004/0010036 A1
(43) Pub. Date: Jan. 15, 2004

(54) N,N, -Substituted s-Aryl-Isothioureas, N,N,N, -Substituted s-Aryl-Isothioureas and combinatorial libraries thereof

(76) Inventors: Boris Rogovoy, Gainesville, FL (US); Vladimir Vvedenskiy, San Diego, CA (US); Xiaohong Cai, Gainesville, FL (US); Christophe Chassaing, Vienna (AT); Alan R. Katritzky, Gainesville, FL (US); Behrouz Forood, Encinitas, CA (US)

Correspondence Address: MCDERMOTT, WILL & EMERY, 600 13TH STREET, N.W., WASHINGTON, DC, 20005-3096, US

(21) Appl. No.: 10/139,646

(22) Filed: May 7, 2002

(51) Int. Cl. A61K 31/215; C07C 335/00; G01N 33/53; G01N 33/543

(52) U.S. Cl. 514/508; 435/7.1; 436/518; 558/4

(57) ABSTRACT

The present invention relates to novel N,N,-Substituted S-Aryl-Isothioureas, and N,N,N′-Substituted S-Aryl-Isothioureas and combinatorial libraries thereof, as well as methods of preparing N,N,-Substituted S-Aryl-Isothioureas, and N,N,N′-Substituted S-Aryl-Isothiourea derivative compounds.

### FIELD OF THE INVENTION

The present invention relates generally to the synthesis of compounds comprising thiourea. In one embodiment, the invention provides novel S-aryl-isothiourea derivative compounds as well as novel combinatorial libraries comprised of such compounds.

### BACKGROUND INFORMATION

The process of discovering new therapeutically active compounds for a given indication involves the screening of all compounds from available compound collections. From the compounds tested, one or more structures are selected as a promising lead. A large number of related analogs are then synthesized in order to develop a structure-activity relationship and select one or more optimal compounds. With traditional “one-at-a-time” synthesis and biological testing of analogs, this optimization process is long and labor intensive. Adding significant numbers of new structures to the compound collections used in the initial screening step of the discovery and optimization process cannot be accomplished with traditional “one-at-a-time” synthesis methods, except over a time frame of years or even decades. Faster methods are needed that allow for the preparation of up to thousands of related compounds in a matter of days or a few weeks. This need is particularly evident when it comes to synthesizing more complex compounds, such as S-Aryl-isothiourea derivative compounds.

Combinatorial approaches have been extended to “organic,” or non-peptide, libraries. However, the libraries to date contain isothiourea compounds of limited diversity and complexity. Such compounds are particularly limited regarding the variations in the N and S substituents. The primary advantage of the present method is the ability to vary the S-aryl substituent. There are only a few examples in the prior art with strongly electron deficient aryl groups (e.g. Ried, W., Erle, H. E., N-Acylisothioureas from N-acylchloroformamidine, Justus Liebigs Ann. Chem. [JLACBF] 1982, 201; Vowinkel, E., Claussen, G., Chem. Ber. [CHBEAM], 1974, 107, 898).

A need therefore exists to develop more complex libraries based on medicinal compounds which would need less time and effort in the synthesis and testing required to bring an organic pharmaceutical product to fruition. In short, improved methods for generating therapeutically useful compounds, such as isothiourea derivatives, are desired.

Thiourea and isothiourea derivative compounds have been the subject of investigation in a number of different biological areas. For example, thiourea derivatives have been proposed or used as an antiviral agent (U.S. Pat. No. 6,300,351, Vig , et al. issued Oct. 9, 2001; U.S. Pat. No. 6,258,831, Camden, issued Jul. 10, 2001), an immunosuppressant agent (U.S. Pat. No. 4,649,138, Durant, et al., issued Mar. 10, 1987), and for use in the treatment of cerebral ischaemia (U.S. Pat. No. 6,090,846, Oplinger, et al., issued Jul. 18, 2000).

Isothiourea derivatives are also known as chemical intermediates in the preparation of guanidine derivatives (see U.S. Pat. No. 4,211,867, Rasmussen, issued Jul. 8, 1980) and Synthesis (1988) 6, 460-466 (Rasmussen) which disclose the compound 4-dimethylaminophenylcarbamimidothioic acid methyl ester and U.S. Pat. No. 5,223,498, Gopalan, issued Jun. 29, 1993.

However, as discussed above, more complex isothiourea derivatives, especially those with variations in the S-aryl substituent have been difficult to attain using known methods.

This invention satisfies this need and provides related advantages as well. The present invention overcomes the known limitations to classical serial organic synthesis of isothiourea derivatives, for example, as well as the shortcomings of combinatorial chemistry related to isothiourea derivatives. The present invention allows for rapid generation of large diverse libraries of complex isothiourea derivatives as discrete molecules. The present invention can utilize a readily available pool of building blocks that can be incorporated into the various regions of the molecule. Furthermore, the method of making the present invention allows for the use of building blocks that contain a wide range of diverse functionality. Such building blocks can provide combinatorial libraries that consist of large numbers as well as combinatorial libraries that are extremely diverse with respect to the functionality contained within those libraries. The present invention combines the techniques of solid-phase synthesis of isothiourea derivatives and the general techniques of synthesis of combinatorial libraries to prepare highly diverse new isothiourea derivative compounds.

| R1 | MDLR1 | R2 | MDLR2 | R3 | MDLR3 | R4 | MDLR4 |
|---|---|---|---|---|---|---|---|
| 4-HYDROXY- | MFCD00- | 4-METHYLDENZYLAMINE | MFCD00008123 | 2,4-DIMETHYL- | MFD00010019 | 2,4-DIFLUOROPHENYL ISOCYANATE | MFCD0001997 |
| BENZALDEHYDE | 006939 | | | THIOPHENOL | | | |
| 3-HYDROXY | MFCD00- | 3,4-DICHLOROBENZYLAMINE | MFCD00008114 | 3-METHOXY- | MFCD00004841 | 2-FLUOROPHENYL ISOCYANATE | MFCD0001196 |
| | | | | THIOPHENOL | | | |
| BENZALDEHYDE | 003368 | 2-METHOXYETHYLAMINE | MFCD00008180 | M-THIOCRESOL | MFCD00004843 | 2-METHOXY-5-METHYLPHENYL ISOCYANATE | MFCD00013869 |
| | | 3-CHLOROBENZYLAMINE | MFCD00040752 | P-THIOCRESOL | MFCD00004851 | 2-NITROPHENYL ISOCYANATE | MFCD00007092 |
| | | ISOBUTYLAMINE | MFCD00008146 | THIOPHENOL | MFCD00004826 | 3-(METHYLTHIO)PHENYL ISOCYANATE | MFCD00013863 |
| | | 2-(4-CHLOROPHENYL)ETHYLAMINE | MFCD00008191 | | | 3-(TRIFLUOROMETHYL)PHENYL ISOCYANATE | MFCD00002020 |
| | | 3-METHOXYBENZYLAMINE | MFCD00008115 | | | 3,4-DICHLOROPHENYL ISOCYANATE | MFCD00002017 |
| | | BENZYLAMINE | MFCD00008106 | | | 3,4-DIFLUOROPHENYL ISOCYANATE | MFCD00065711 |
| | | BUTYLAMINE | MFCD0011690 | | | 3,4-DIMETHYLPHENYL ISOCYANATE | MFCD00013867 |
| | | 2-(3-METHOXYPHENYL)ETHYLAMINE | MFCD00008187 | | | 3,5-DICHLOROPHENYL ISOCYANATE | MFCD00013859 |
| | | 2-(4-FLUOROPHENYL)ETHYLAMINE | MFCD00134208 | | | 3,5-DIMETHOXYPHENYL ISOCYANATE | MFCD00013862 |
| | | 4-METHOXYBENZYLAMINE | MFCD00008122 | | | 3,5-DIMETHYLPHENYL ISOCYANATE | MFCD00013868 |
| | | 2-ETHOXYETHYLAMINE | MFCD00025604 | | | 3-CHLORO-4-FLUOROPHENYL ISOCYANATE | MFCD00037037 |
| | | AMINOMETHYLCYCLOHEXANE | MFCD00001520 | | | 3-CHLORO-4-METHYLPHENYL ISOCYANATE | MFCD0013858 |
| | | CYCLOHEXYLAMINE | MFCD00001486 | | | 3-CHLOROPHENYL ISOCYANATE | MFCD00002016 |
| | | 3,4-DIMETHOXYBENZYLAMINE | MFCD00008116 | | | 3-ETHYLPHENYL ISOCYANATE | MFCD00013870 |
| | | 2-(4-METHOXYPHENYL)ETHYLAMINE | MFCD00008192 | | | 3-FLUORO-4-METHYLPHENYL ISOCYANATE | MFCD00037071 |
| | | DL-ALPHA-METHYLBENZYLAMINE | MFCD00008069 | | | 3-FLUOROPHENYL ISOCYANATE | MFCD00002015 |
| | | | | | | 3-METHOXYPHENYL ISOCYANATE | MFCD00002019 |
| | | | | | | 3-METHYLBENZYL ISOCYANATE | MFCD00673059 |
| | | | | | | 3-NITROPHENYL ISOCYANATE | MFCD00013878 |
| | | | | | | 3-NITROPHENYL ISOCYANATE | MFCD00007220 |
| | | | | | | 4-(METHYLTHIO)PHENYL ISOCYANATE | MFCD00035702 |
| | | | | | | 4-(TRIFLUOROMETHOXY)PHENYL ISOCYANATE | MFCD00035702 |
| | | | | | | 4-(TRIFLUOROMETHYL)PHENYL ISOCYANATE | MFCD00002028 |
| | | | | | | 4-BIPHENYLYL ISOCYANATE | MFCD00037089 |
| | | | | | | 4-BROMOPHENYL ISOCYANATE | MFCD00002022 |
| | | | | | | 4-BUTOXYPHENYL ISOCYANATE | MFCD00037042 |
| | | | | | | 4-CHLORO-3-(TRIFLUOROMETHYL) | MFCD00013874 |
| | | | | | | PHENYL ISOCYANATE | |
| | | | | | | 4-CHLORO-3-NITROPHENYL ISOCYANATE | MFCD00037062 |
| | | | | | | 4-CHLOROPHENYL ISOCYANATE | MFCD00002024 |
| | | | | | | 4-CYANOPHENYLISOCYANATE | MFCD00037038 |
| | | | | | | 4-ETHOXYPHENYL ISOCYANATE | MFCD00013877 |
| | | | | | | 4-ETHYLPHENYL ISOCYANATE | MFCD00013881 |
| | | | | | | 4-FLUORO-3-NITROPHENYL ISOCYANATE | MFCD00007055 |
| | | | | | | 4-FLUOROBENZYL ISOCYANATE | MFCD00673062 |
| | | | | | | 4-ISOPROPYLPHENYL ISOCYANATE | MFCD00013880 |
| | | | | | | 4-METHOXYPHENYL ISOCYANATE | MFCD00002026 |
| | | | | | | 4-N-BUTYLPHENYL ISOCYANATE | MFCD00013882 |
| | | | | | | BENZYL ISOCYANATE | MFCD00009701 |
| | | | | | | P-TOLYL ISOCYANATE | MFCD00002029 |

### SUMMARY OF THE INVENTION

The present invention relates to novel isothiourea derivative compounds of the following formula:

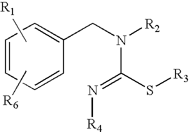

wherein

R₁ and R₆ are independently selected from the group consisting of H, —OH, halide, C₁ to C₇ alkoxy, substituted C₁ to C₇ alkoxy, amino, substituted amino, acylamino, substituted acylamino, carboxyl, carboxylate ester, carboxamide, substituted carboxamide, phenyl, substituted phenyl, C₁ to C₆ alkyl, C₁ to C₆ substituted alkyl, C₁ to C₆ alkenyl, C₁ to C₆ substituted alkenyl, C₁ to C₆ alkynyl, C₁ to C₆ substituted alkynyl, nitro, thiol, thioether, and substituted thioether, or R1 and R6 taken together form a ring fused to the phenyl to form a naphthyl ring,

R₂ is selected from the group consisting of C₁ to C₆ alkyl, C₁ to C₆ substituted alkyl, benzyl, substituted benzyl, phenylalkyl and substituted phenylalkyl,

R3 is selected from the group of phenyl, substituted phenyl, naphthyl and substituted naphthyl; and

R₄ is selected from the group consisting of H, and an unsubstituted or a substituted phenyl, wherein said substituted phenyl is substituted with at least one substituent selected from the group consisting of: halo, C₁ to C₆ alkyl, C₁ to C₆ alkoxy, nitro, C₁ to C₆ substituted alkyl, C₁ to C₆ substituted alkoxy, C₁ to C₆ alkylthio, phenyl, cyano, benzyl, substituted benzyl, naphthyl, and substituted naphthyl.

The invention further relates to combinatorial libraries containing two or more such compounds, as well as methods of preparing isothiourea derivative compounds.

### BRIEF DESCRIPTION OF THE DRAWING

FIG. 1 shows a scheme for the combinatorial synthesis of isothiourea derivative compounds.

### DETAILED DESCRIPTION OF THE INVENTION

The present invention provides compounds and combinatorial libraries of compounds of the formula:

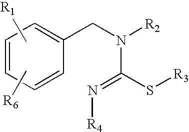

R₁ and R₆ are independently selected from the group consisting of H, —OH, halide, C₁ to C₇ alkoxy, substituted C₁ to C₇ alkoxy, amino, substituted amino, acylamino, substituted acylamino, carboxyl, carboxylate ester, carboxamide, substituted carboxamide, phenyl, substituted phenyl, C₁ to C₆ alkyl, C₁ to C₆ substituted alkyl, C₁ to C₆ alkenyl, C₁ to C₆ substituted alkenyl, C₁ to C₆ alkynyl, C₁ to C₆ substituted alkynyl, nitro, thiol, thioether, and substituted thioether, or R1 and R6 taken together form a ring fused to the phenyl to form a naphthyl ring,

R₂ is selected from the group consisting of C₁ to C₆ alkyl, C₁ to C₆ substituted alkyl, benzyl, substituted benzyl, phenylalkyl and substituted phenylalkyl,

R3 is selected from the group of phenyl, substituted phenyl, naphthyl and substituted naphthyl; and

R4 is selected from the group consisting of H, and an unsubstituted or a substituted phenyl, wherein said substituted phenyl is substituted with at least one substituent selected from the group consisting of: halo, C₁ to C₆ alkyl, C₁ to C₆ alkoxy, nitro, C₁ to C₆ substituted alkyl, C₁ to C₆ substituted alkoxy, C₁ to C₆ alkylthio, phenyl, cyano, benzyl, substituted benzyl, naphthyl, and substituted naphthyl.

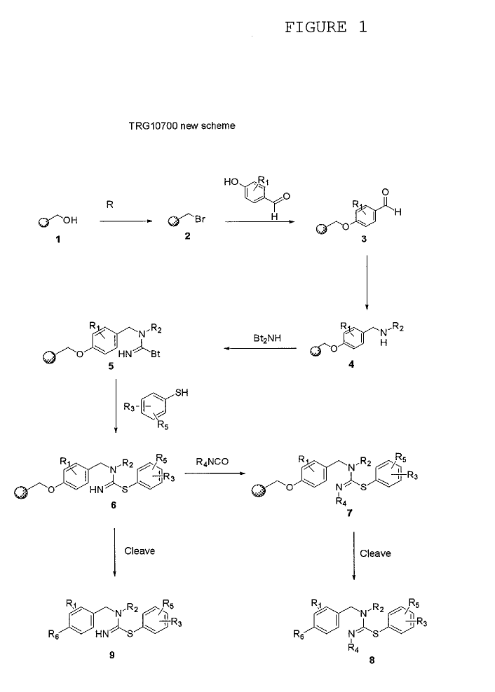

The invention also provides methods of preparing isothiourea derivative compounds and combinatorial libraries. In one method, as shown in FIG. 1, such compounds can be prepared by a process comprising:

preparing a resin bound linker,

reacting said resin bound linker with an aldehyde substituted compound to produce a resin bound aldehyde compound,

reacting said resin bound aldehyde with an amine to produce a resin bound amine,

reacting said resin bound amine with di(benzotriazole-1-yl)methanimine to produce a resin bound benzotriazole amidine,

reacting said resin bound benzotriazole amidine with a thiophenol to produce a resin bound N,N-substituted N′ unsubstituted S-Aryl isothiourea, and

cleaving and extracting said N,N-substituted S-Aryl isothiourea from said resin or reacting said resin bound N,N-substituted N′ unsubstituted S-Aryl isothiourea with an isocyanate to produce an N,N,N′ substituted S-Aryl isothiourea resin, and then cleaving and extracting said N,N,N′ substituted S-Aryl isothiourea from said resin.

When the above-described compounds include one or more chiral centers, the stereochemistry of such chiral centers can independently be in the R or S configuration, or a mixture of the two. The chiral centers can be further designated as R or S or R,S or d,D, l,L or d,l, D,L.

In the above formula , the term “C₁ to C₆ alkyl” denotes such radicals as methyl, ethyl, n-propyl, isopropyl, n-butyl, iso-butyl, sec-butyl, tert-butyl, amyl, tert-amyl, hexyl and the like. The preferred “C₁ to C₆ alkyl” groups are methyl, iso-butyl, sec-butyl and iso-propyl.

The term “C₁ to C₆ substituted alkyl,” denotes that the above C₁ to C₆ alkyl groups are substituted by one or more, and preferably one or two, halogen, hydroxy, protected hydroxy, oxo, protected oxo, C₃ to C₇ cycloalkyl, naphthyl, amino, protected amino, (monosubstituted)amino, protected (monosubstituted) amino, (disubstituted) amino, guanidino, protected guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, carboxy, protected carboxy, carbamoyl, carboxamide, protected carboxamide, N-(C₁ to C₆ alkyl)carboxamide, protected N-(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted alkyl groups may be substituted once or more, and preferably once or twice, with the same or with different substituents.

Examples of the above substituted alkyl groups include the 2-oxo-prop-1-yl, 3-oxo-but-1-yl, cyanomethyl, nitromethyl, chloromethyl, hydroxymethyl, tetrahydropyranyloxymethyl, trityloxymethyl, propionyloxymethyl, amino, methylamino, aminomethyl, dimethylamino, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl, N-acetyl-1-aminoethyl and the like.

The term “C₁ to C₇ alkoxy” as used herein denotes groups such as methoxy, ethoxy, n-propoxy, isopropoxy, n-butoxy, t-butoxy and like groups. A preferred alkoxy is methoxy. The term “C₁ to C₇ substituted alkoxy” means the alkyl portion of the alkoxy can be substituted in the same manner as in relation to C₁ to C₆ substituted alkyl. Similarly, the term “C₁ to C₇ phenylalkoxy” as used herein means “C₁ to C₇ alkoxy” bonded to a phenyl radical.

The substituent term “C₃ to C₇ cycloalkyl” includes the cyclopropyl, cyclobutyl, cyclopentyl, cyclohexyl or cycloheptyl rings. The substituent term “C₃ to C₇ substituted cycloalkyl” indicates the above cycloalkyl rings substituted by one or two halogen, hydroxy, protected hydroxy, C₁ to C₄ alkylthio, C₁ to C₄ alkylsulfoxide, C₁ to C₄ alkylsulfonyl, C₁ to C₄ substituted alkylthio, C₁ to C₄ substituted alkylsulfoxide, C₁ to C₄ substituted alkylsulfonyl, C₁ to C₆ alkyl, C₁ to C₇ alkoxy, C₁ to C₆ substituted alkyl, C₁ to C₇ alkoxy, oxo, protected oxo, (monosubstituted) amino, (disubstituted) amino, trifluoromethyl, carboxy, protected carboxy, phenyl, substituted phenyl, phenylthio, phenylsulfoxide, phenylsulfonyl, amino, or protected amino groups.

The term “substituted phenyl” specifies a phenyl group substituted with one or more, and preferably one or two, moieties chosen from the groups consisting of halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ alkylthio, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl)carboxamide, protected N-(C₁ to C₆ alkyl)carboxamide, N, N-di(C₁ to C₆ alkyl)carboxamide, trifluoromethyl, N-((C₁ to C₆ alkyl)sulfonyl)amino, -(phenylsulfonyl)amino or phenyl, wherein the phenyl is substituted or unsubstituted, such that, for example, a biphenyl results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3 or 4-hydroxyphenyl, 2,4-dihydroxyphenyl, the protected-hydroxy derivatives thereof and the like; a nitrophenyl group such as 2, 3 or 4-nitrophenyl; a cyanophenyl group, for example, 2, 3 or 4-cyanophenyl; a mono- or di(alkyl)phenyl group such as 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl and the like; a mono or di(alkoxyl)phenyl group, for example, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono-or di(hydroxymethyl)phenyl or (protected hydroxymethyl)phenyl such as 2, 3, or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like.

The terms “C₂ to C₆ substituted alkenyl, ” and “C₁ to C₆ substituted alkynyl,” denote groups that are substituted by one or more, and preferably one or two, of halogen, hydroxy, protected hydroxy, oxo, protected oxo, C₃ to C₇ cycloalkyl, phenyl, naphthyl, amino, protected amino, (monosubstituted)amino, protected (monosubstituted)amino, (disubstituted)amino, guanidino, protected guanidino, heterocyclic ring, substituted heterocyclic ring, imidazolyl, indolyl, pyrrolidinyl, C₁ to C₁₂ alkoxy, C₁ to C₂₀ acyl, C₁ to C₂₀ acyloxy, nitro, carboxy, protected carboxy, carbamoyl, carboxamide, protected carboxamide, N-(C₁ to C₂₀ alkyl)carboxamide, protected N-(C₁ to C₂₀ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted groups may be substituted once or more, and preferably once or twice, with the same or with different substituents.

Examples of the above substituted alkenyl groups include styrenyl, 3-chloro-propen-1-yl, 3-chloro-buten-1-yl, 3-methoxy-propen-2-yl, 3-phenyl-buten-2-yl, 1-cyano-buten-3-yl and the like. The geometrical isomerism is not critical, and all geometrical isomers for a given substituted alkenyl can be used.

Examples of the above substituted alkynyl groups include phenylacetylen-1-yl, 1-phenyl-2-propyn-1-yl and the like.

The terms “C₇ to C₁₈ substituted phenylalkyl” and “C₁ to C₁₂ substituted heterocycloalkyl” denote a C₇ to C₁₈ phenylalkyl group or C₁ to C₁₂ heterocycloalkyl substituted (on the alkyl or, where applicable, phenyl or heterocyclic portion) with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, protected guanidino, heterocyclic ring, substituted heterocyclic ring, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, nitro, carboxy, protected carboxy, carbamoyl, carboxamide, protected carboxamide, N-(C₁ to C₁₂ alkyl)carboxamide, protected N-(C₁ to C₁₂ alkyl)carboxamide, N, N-(C₁ to C₁₂ dialkyl) carboxamide, cyano, N-(C₁ to C₁₂ alkylsulfonyl)amino, thiol, C₁ to C₁₀ alkylthio, C₁ to C₁₀ alkylsulfonyl groups; and/or the phenyl group may be substituted with one or more, and preferably one or two, substituents chosen from halogen, hydroxy, protected hydroxy, cyano, nitro, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted) amino, (disubstituted) amino, carboxamide, protected carboxamide, N-(C₁ to C₁₂ alkyl)carboxamide, protected N-(C₁ to C₁₂ alkyl)carboxamide, N, N-di(C₁ to C₁₂ alkyl)carboxamide, trifluoromethyl, N-((C₁ to C₁₂ alkyl) sulfonyl) amino, N-(phenylsulfonyl) amino, cyclic C₂ to C₁₂ alkylene or a phenyl group, substituted or unsubstituted, for a resulting biphenyl group. The substituted alkyl, phenyl or heterocyclic groups may be substituted with one or more, and preferably one or two, substituents which can be the same or different.

Examples of the term “C₇ to C₁₈ substituted phenylalkyl” include groups such as 2-phenyl-1-chloroethyl, 2-(4-methoxyphenyl)ethyl, 4-(2,6-dihydroxy phenyl)n-hexyl, 2-(5-cyano-3-methoxyphenyl)n-pentyl, 3-(2,6-dimethylphenyl)n-propyl, 4-chloro-3-aminobenzyl, 6-(4-methoxyphenyl)-3-carboxy(n-hexyl), 5-(4-aminomethylphenyl)-3-(aminomethyl)n-pentyl, 5-phenyl-3-oxo-n-pent-1-yl and the like.

The terms “halo”, “halide” and “halogen” refer to the fluoro, chloro, bromo or iodo atoms. There can be one or more halogen, which are the same or different. Preferred halogens are chloro and fluoro.

The term “(monosubstituted)amino” refers to an amino group with one substituent chosen from the group consisting of phenyl, substituted phenyl, C₁ to C₆ alkyl, C₁ to C₆ substituted alkyl, C₁ to C₇ acyl, C₁ to C₇ substituted acyl, C₂ to C₇ alkenyl, C₂ to C₇ substituted alkenyl, C₂ to C₇ alkynyl, C₂ to C₇ substituted alkynyl, C₇ to C₁₂ phenylalkyl, C₇ to C₁₂ substituted phenylalkyl and heterocyclic ring. The (monosubstituted)amino can additionally have an amino-protecting group as encompassed by the term “protected (monosubstituted)amino.”

The term “(disubstituted)amino” refers to an amino group with two substituents chosen from the group consisting of phenyl, substituted phenyl, C₁ to C₆ alkyl, C₁ to C₆ substituted alkyl, C₁ to C₇ acyl, C₂ to C₇ alkenyl, C₂ to C₇ alkynyl, C₇ to C₁₂ phenylalkyl, and C₇ to C₁₂ substituted phenylalkyl. The two substituents can be the same or different.

The term “C₁ to C₄ alkylthio” refers to sulfide groups such as methylthio, ethylthio, n-propylthio, isopropylthio, n-butylthio, t-butylthio and like groups.

The term “C₁ to C₄ substituted alkylthio, ”denotes that the C₁ to C₄ alkyl portion of this group may be substituted as described above in relation to “substituted alkyl.”

The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted) amino, carboxamide, protected carboxamide, N-(C₁ to C₁₂ alkyl)carboxamide, protected N-(C₁ to C₁₂ alkyl)carboxamide, N, N-di(C₁ to C₁₂ alkyl)carboxamide, trifluoromethyl, N-((C₁ to C₁₂ alkyl) sulfonyl)amino or N-(phenylsulfonyl) amino.

Examples of the term “substituted naphthyl” includes a mono or di(halo)naphthyl group such as 1, 2, 3, 4, 5, 6, 7 or 8-chloronaphthyl, 2, 6-dichloronaphthyl, 2, 5-dichloronaphthyl, 3, 4-dichloronaphthyl, 1, 2, 3, 4, 5, 6, 7 or 8-bromonaphthyl, 3, 4-dibromonaphthyl, 3-chloro-4-fluoronaphthyl, 1, 2, 3, 4, 5, 6, 7 or 8-fluoronaphthyl and the like; a mono or di(hydroxy)naphthyl group such as 1, 2, 3, 4, 5, 6, 7 or 8-hydroxynaphthyl, 2, 4-dihydroxynaphthyl, the protected-hydroxy derivatives thereof and the like; a nitronaphthyl group such as 3- or 4-nitronaphthyl; a cyanonaphthyl group, for example, 1, 2, 3, 4, 5, 6, 7 or 8-cyanonaphthyl; a mono- or di(alkyl)naphthyl group such as 2, 3, 4, 5, 6, 7 or 8-methylnaphthyl, 1, 2, 4-dimethylnaphthyl, 1, 2, 3, 4, 5, 6, 7 or 8-(isopropyl)naphthyl, 1, 2, 3, 4, 5, 6, 7 or 8-ethylnaphthyl, 1, 2, 3, 4, 5, 6, 7 or 8-(n-propyl)naphthyl and the like; a mono or di(alkoxy)naphthyl group, for example, 2, 6-dimethoxynaphthyl, 1, 2, 3, 4, 5, 6, 7 or 8-methoxynaphthyl, 1, 2, 3, 4, 5, 6, 7 or 8-ethoxynaphthyl, 1, 2, 3, 4, 5, 6, 7 or 8-(isopropoxy)naphthyl, 1, 2, 3, 4, 5, 6, 7 or 8-(t-butoxy)naphthyl, 3-ethoxy-4-methoxynaphthyl and the like; 1, 2, 3, 4, 5, 6, 7 or 8-trifluoromethylnaphthyl; a mono- or dicarboxynaphthyl or (protected carboxy)naphthyl group such as 1, 2, 3, 4, 5, 6, 7 or 8-carboxynaphthyl or 2, 4-di(-protected carboxy)naphthyl; a mono-or di(hydroxymethyl)naphthyl or (protected hydroxymethyl)naphthyl such as 1, 2, 3, 4, 5, 6, 7 or 8-(protected hydroxymethyl)naphthyl or 3, 4-di(hydroxymethyl)naphthyl; a mono- or di(amino)naphthyl or (protected amino)naphthyl such as 1, 2, 3, 4, 5, 6, 7 or 8-(amino)naphthyl or 2, 4-(protected amino)-naphthyl, a mono- or di(aminomethyl)naphthyl or (protected aminomethyl)naphthyl such as 2, 3, or 4-(aminomethyl)naphthyl or 2, 4-(protected aminomethyl)-naphthyl; or a mono- or di-(N-methylsulfonylamino) naphthyl such as 1, 2, 3, 4, 5, 6, 7 or 8-(N-methylsulfonylamino)naphthyl. Also, the term “substituted naphthyl” represents disubstituted naphthyl groups wherein the substituents are different, for example, 3-methyl-4-hydroxynaphth-1-yl, 3-chloro-4-hydroxynaphth-2-yl, 2-methoxy-4-bromonaphth-1-yl, 4-ethyl-2-hydroxynaphth-1-yl, 3-hydroxy-4-nitronaphth-2-yl, 2-hydroxy-4-chloronaphth-1-yl, 2-methoxy-7-bromonaphth-1-yl, 4-ethyl-5-hydroxynaphth-2-yl, 3-hydroxy-8-nitronaphth-2-yl, 2-hydroxy-5-chloronaphth-1-yl and the like.

The term “C₁ to C₁₀ substituted thioether,” denotes a C₁ to C₁₀ substituted alkylthio or phenylthio group where the C₁ to C₁₀ alkyl portion of these groups may be substituted as described above in relation to “substituted alkyl,” and that the phenyl ring of these groups can be substituted as described above in relation to “substituted phenyl.”

The term “substituted carboxamide” refers to a carboxamide group with the Nitrogen having one or two substituents chosen from the group consisting of phenyl, substituted phenyl, C₁ to C₁₂ alkyl, C₁ to C₁₂ substituted alkyl, C₂ to C₁₂ alkenyl, C₂ to C₁₂ substituted alkenyl, C₂ to C₁₂ alkynyl, C₂ to C₁₂ substituted alkynyl, C₇ to C₁₈ phenylalkyl, C₇ to C₁₈ substituted phenylalkyl, heterocyclic ring, substituted heterocyclic ring, C₁ to C₁₂ heterocycloalkyl and C₁ to C₁₂ substituted heterocycloalkyl. The two substituents can be the same or different.

The terms “linker” and “Wang linker” denotes any compound with a suitable functional group.

One or more of the compounds of the invention, even within a given library, may be present as a salt (e.g. isothiourea can be in the form of an isothiouronium salt). The term “salt” encompasses those salts that form with the carboxylate anions and amine nitrogens and include salts formed with the organic and inorganic anions and cations discussed below. Furthermore, the term includes salts that form by standard acid-base reactions with basic groups (such as amino groups) and organic or inorganic acids. Such acids include hydrochloric, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977). Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. For example, a cation for a carboxylate anion will exist when R₂ or R₃ is substituted with a (quaternary ammonium)methyl group. A preferred cation for the carboxylate anion is the sodium cation.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

One or more compounds of the invention, even when in a library, can be in the biologically active ester form, such as the non-toxic, metabolically-labile ester-form. Such ester forms induce increased blood levels and prolong the efficacy of the corresponding non-esterified forms of the compounds. Ester groups which can be used include the lower alkoxymethyl groups, for example, methoxymethyl, ethoxymethyl, isopropoxymethyl and the like; the -(C₁ to C₇) alkoxyethyl groups, for example methoxyethyl, ethoxyethyl, propoxyethyl, isopropoxyethyl and the like; the 2-oxo-1,3-diooxlen-4-ylmethyl groups, such as 5-methyl-2-oxo-1,3-dioxolen-4-ylmethyl, 5-phenyl-2-oxo-1,3-dioxolen-4-ylmethyl and the like; the C₁ to C₄ alkylthiomethyl groups, for example methylthiomethyl, ethylthiomethyl, iso-propylthiomethyl and the like; the acyloxymethyl groups, for example pivaloyloxymethyl, pivaloyloxyethyl, -acetoxymethyl and the like; the ethoxycarbonyl-l-methyl group; the —acetoxyethyl; the 1-(C₁ to C₇ alkyloxycarbonyloxy)ethyl groups such as the 1-(ethoxycarbonyloxy)ethyl group; and the 1-(C₁ to C₇ alkylaminocarbonyloxy)ethyl groups such as the 1-(methylaminocarbonyloxy)ethyl group.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nvav”), L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984. Amino acids and amino acid analogs can be purchased commercially (Sigma Chemical Co.; Advanced Chemtech) or synthesized using methods known in the art.

The term “functionalized resin” means any resin, crosslinked or otherwise, where functional groups have been introduced into the resin, as is common in the art. Such resins include, for example, those functionalized with amino, alkylhalo, formyl or hydroxy groups. Such resins which can serve as solid supports are well known in the art and include, for example, 4-methylbenzhydrylamine-copoly(styrene-1% divinylbenzene) (MBHA), 4-hydroxymethylphenoxymethyl-copoly(styrene-1% divinylbenzene), 4-oxymethyl-phenyl-acetamido-copoly(stryene-1% divinylbenzene)(Wang), 4-(oxymethyl)-phenylacetamido methyl (Pam), and Tentage™, from Rapp Polymere Gmbh, trialkoxy-diphenyl-methyl ester-copoly(styrene-1% divinylbenzene)(RINK) all of which are commercially available. Other functionalized resins are known in the art and can be use without departure from the scope of the current invention. Such resins may include those described in Jung, G., Combinatorial Peptide and Nonpeptide Libraties, A Handbook (VCH Verlag, 1996) or Bunin, B. A., The Combinatorial Index (Academic Press, 1998).

As used herein, a “combinatorial library” is an intentionally created collection of differing molecules which can be prepared by the means provided below or otherwise and screened for biological activity in a variety of formats (e.g., libraries of soluble molecules, libraries of compounds attached to resin beads, silica chips or other solid supports). A “combinatorial library,” as defined above, involves successive rounds of chemical syntheses based on a common starting structure. The combinatorial libraries can be screened in any variety of assays, such as those detailed below as well as others useful for assessing their biological activity. The combinatorial libraries will generally have at least one active compound and are generally prepared such that the compounds are in equimolar quantities.

A combinatorial library of the invention can contain one or more of the above-described compounds. The invention further provides a combinatorial library containing five or more of the above-described compounds. In another embodiment of the invention, a combinatorial library can contain ten or more of the above-described compounds. In yet another embodiment of the invention, a combinatorial library can contain fifty or more of the above-described compounds. If desired, a combinatorial library of the invention can contain 100,000 or more, or even 1,000,000 or more, of the above-described compounds.

By way of example, the preparation of the combinatorial libraries can use the “split resin approach.” The split resin approach is described by, for example, U.S. Pat. No. 5,010,175 to Rutter, WO PCT 91/19735 to Simon, and Gallop et al., J. Med. Chem., 37:1233-1251 (1994).

For preparing pharmaceutical compositions containing compounds of the invention, inert, pharmaceutically acceptable carriers are used. The pharmaceutical carrier can be either solid or liquid. Solid form preparations include, for example, powders, tablets, dispersible granules, capsules, cachets, and suppositories.

A solid carrier can be one or more substances which can also act as diluents, flavoring agents, solubilizers, lubricants, suspending agents, binders, or tablet disintegrating agents; it can also be an encapsulating material.

In powders, the carrier is generally a finely divided solid which is in a mixture with the finely divided active component. In tablets, the active compound is mixed with the carrier having the necessary binding properties in suitable proportions and compacted in the shape and size desired.

For preparing pharmaceutical composition in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter and the like.

The pharmaceutical compositions can include the formulation of the active compound with encapsulating material as a carrier providing a capsule in which the active component (with or without other carriers) is surrounded by a carrier, which is thus in association with it. In a similar manner, cachets are also included. Tablets, powders, cachets, and capsules can be used as solid dosage forms suitable for oral administration.

Liquid pharmaceutical compositions include, for example, solutions suitable for oral or parenteral administration, or suspensions, and emulsions suitable for oral administration. Sterile water solutions of the active component or sterile solutions of the active component in solvents comprising water, ethanol, or propylene glycol are examples of liquid compositions suitable for parenteral administration.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Aqueous solutions for oral administration can be prepared by dissolving the active compound in water and adding suitable flavorants, coloring agents, stabilizers, and thickening agents as desired. Aqueous suspensions for oral use can be made by dispersing the finely divided active component in water together with a viscous material such as natural or synthetic gums, resins, methyl cellulose, sodium carboxymethyl cellulose, and other suspending agents known to the pharmaceutical formulation art.

Preferably, the pharmaceutical composition is in unit dosage form. In such form, the composition is divided into unit doses containing appropriate quantities of the active isothiourea. The unit dosage form can be a packaged preparation, the package containing discrete quantities of the preparation, for example, packeted tablets, capsules, and powders in vials or ampules. The unit dosage form can also be a capsule, cachet, or tablet itself, or it can be the appropriate number of any of these packaged forms.

As pharmaceutical compositions for treating infections, pain, or any other indication the compounds of the present invention are generally in a pharmaceutical composition so as to be administered to a subject at dosage levels of from 0.7 to 7000 mg per day, and preferably 1 to 500 mg per day, for a normal human adult of approximately 70 kg of body weight, this translates into a dosage of from 0.01 to 100 mg/kg of body weight per day. The specific dosages employed, however, can be varied depending upon the requirements of the patient, the severity of the condition being treated, and the activity of the compound being employed. The determination of optimum dosages for a particular situation is within the skill of the art.

Variant isothiourea derivative compounds and combinatorial libraries can be prepared as shown in FIG. 1 in order to achieve a high level of diversity.

The resulting compound can be cleaved from the resin. Resin-bound isothiourea derivative compounds can be cleaved by treating them, for example, with HF. They can also be cleaved with TFA/DCM, when a TFA sensitive linker is used. The compounds can be extracted from the spent resin, for example, with AcOH, MeOH or Acetonitrile and water.

The nonsupport-bound combinatorial libraries can be screened as single compounds. In addition, the nonsupport-bound combinatorial libraries can be screened as mixtures in solution in assays such as radio-receptor inhibition assays, anti-bacterial assays, anti-fungal assays, calmodulin-dependent phosphodiesterase (CaMPDE) assays and phosphodiesterase (PDE) assays, as described in detail below. Deconvolution of highly active mixtures can then be carried out by iterative or positional scanning methods. These techniques, the iterative approach or the positional scanning approach, can be utilized for finding other active compounds within the combinatorial libraries of the present invention using any one of the below-described assays or others well known in the art.

The iterative approach is well-known and is set forth in general in Houghten et al., Nature, 354, 84-86 (1991) and Dooley et al., Science, 266, 2019-2022 (1994), both of which are incorporated herein by reference. In the iterative approach, for example, sub-libraries of a molecule having three variable groups are made wherein the first variable is defined. Each of the compounds with the defined variable group is reacted with all of the other possibilities at the other two variable groups. These sub-libraries are each tested to define the identity of the second variable in the sub-library having the highest activity in the screen of choice. A new sub-library with the first two variable positions defined is reacted again with all the other possibilities at the remaining undefined variable position. As before, the identity of the third variable position in the sub-library having the highest activity is determined. If more variables exist, this process is repeated for all variables, yielding the compound with each variable contributing to the highest desired activity in the screening process. Promising compounds from this process can then be synthesized on larger scale in traditional single-compound synthetic methods for further biological investigation.

The positional-scanning approach has been described for various combinatorial libraries as described, for example, in R. Houghten et al. PCT/US91/08694 and U.S. Pat. No. 5,556,762, both of which are incorporated herein by reference. In the positional scanning approach, sublibraries are made defining only one variable with each set of sublibraries and all possible sublibraries with each single variable defined (and all other possibilities at all of the other variable positions), made and tested. From the instant description one skilled in the art could synthesize combinatorial libraries wherein two fixed positions are defined at a time. From the testing of each single-variable defined combinatorial library, the optimum substituent at that position can be determined, pointing to the optimum or at least a series of compounds having a maximum of the desired biological activity. Thus, the number of sublibraries for compounds with a single position defined will be the number of different substituents desired at that position, and the number of all the compounds in each sublibrary will be the product of the number of substituents at each of the other variables.

Individual compounds and pharmaceutical compositions containing the compounds, as well as methods of using the same, are included within the scope of the present invention. The compounds of the present invention can be used for a variety of purposes and indications and as medicaments for any such purposes and indications. For example, S-aryl-isothiourea derivative compounds of the present invention can be used as pesticides, acaricides, receptor agonists or antagonists and antimicrobial agents, including antibacterial or antiviral agents. The libraries can be screened in any variety of melanocortin receptor and related activity assays, such as those detailed below as well as others known in the art. Additionally, the subject compounds can be useful as analgesics. Assays which can be used to test the biological activity of the instant compounds include antimicrobial assays, a competitive enzyme-linked immunoabsorbent assay and radio-receptor assays, as described below.

The melanocortin (MC) receptors are a group of cell surface proteins that mediate a variety of physiological effects, including regulation of adrenal gland function such as production of the glucocorticoids cortisol and aldosterone; control of melanocyte growth and pigment production; thermoregulation; immunomodulation; and analgesia. Five distinct MC receptors have been cloned and are expressed in a variety of tissues, including melanocytes, adrenal cortex, brain, gut, placenta, skeletal muscle, lung, spleen, thymus, bone marrow, pituitary, gonads and adipose tissue (Tatro, Neuroimmunomodulation 3:259-284 (1996)). Three MC receptors, MCR-1, MCR-3 and MCR-4, are expressed in brain tissue (Xia et al., Neuroreport 6:2193-2196 (1995)).

A variety of ligands termed melanocortins function as agonists that stimulate the activity of MC receptors. The melanocortins include melanocyte-stimulating hormones (MSH) such as α-MSH, β-MSH and γ-MSH, as well as adrenocorticotropic hormone (ACTH). Individual ligands can bind to multiple MC receptors with differing relative affinities. The variety of ligands and MC receptors with differential tissue-specific expression likely provides the molecular basis for the diverse physiological effects of melanocortins and MC receptors. For example, α-MSH antagonizes the actions of immunological substances such as cytokines and acts to modulate fever, inflammation and immune responses (Catania and Lipton, Annals N. Y. Acad. Sci. 680:412-423 (1993)).

The role of certain specific MC receptors in some of the physiological effects described above for MC receptors has been elucidated. For example, MCR-1 is involved in pain and inflammation. MCR-1 mRNA is expressed in neutrophils (Catania et al., Peptides 17:675-679 (1996)). The anti-inflammatory agent α-MSH was found to inhibit migration of neutrophils. Thus, the presence of MCR-1 in neutrophils correlates with the anti-inflammatory activity of α-MSH.

An interesting link of MC receptors to regulation of food intake and obesity has recently been described. The brain MC receptor MCR-4 has been shown to function in the regulation of body weight and food intake. Mice in which MCR-4 has been knocked out exhibit weight gain (Huszar et al., Cell 88:131-141 (1997)). In addition, injection into brain of synthetic peptides that mimic melanocortins and bind to MCR-4 caused suppressed feeding in normal and mutant obese mice (Fan et al., Nature 385:165-168 (1997)). These results indicate that the brain MC receptor MCR-4 functions in regulating food intake and body weight.

Due to the varied physiological activities of MC receptors, high affinity ligands of MC receptors could be used to exploit the varied physiological responses of MC receptors by functioning as potential therapeutic agents or as lead compounds for the development of therapeutic agents. Furthermore, due to the effect of MC receptors on the activity of various cytokines, high affinity MC receptor ligands could also be used to regulate cytokine activity.

A variety of assays can be used to identify or characterize MC receptor ligands of the invention. For example, the ability of a isothiourea derivative compound to compete for binding of a known MC receptor ligand can be used to assess the affinity and specificity of a isothiourea derivative compound for one or more MC receptors. Any MC receptor ligand can be used so long as the ligand can be labeled with a detectable moiety. The detectable moiety can be, for example, a radiolabel, fluorescent label or chromophore, or any detectable functional moiety so long as the MC receptor ligand exhibits specific MC receptor binding. A particularly useful detectable MC receptor ligand for identifying and characterizing other MC receptor ligands is 125I-HP 467, which has the amino acid sequence Ac-Nle-Gln-His-(p(I)-D-Phe)-Arg-(D-Trp)-Gly-NH2 and is described in Dooley et al., “Melanocortin Receptor Ligands and Methods of Using Same,” U.S. patent application Ser. No. 09/027,108, filed Feb. 20, 1998, which is incorporated herein by reference. HP 467 is a para-iodinated form of HP 228.

Using assay methods such as those described above, binding kinetics and competition with radiolabeled HP 467 can confirm that isothiourea derivative compounds of the invention bind to one or more MC receptors. Furthermore, S-aryl-isothiourea derivative compounds of the invention can exhibit a range of affinities and specificity for various MC receptors.

The invention provides MC receptor ligands that can bind to several MC receptors with similar affinity. In addition, the invention also provides MC receptor ligands that can be selective for one or more MC receptors. As used herein, the term “selective” means that the affinity of a MC receptor ligand differs between one MC receptor and another by about 10-fold, generally about 20- to 50-fold, and particularly about 100-fold. In some cases, a MC receptor ligand having broad specificity is desired. In other cases, it is desirable to use MC receptor ligands having selectivity for a particular MC receptor. For example, MCR-1 ligands are particularly useful for treating pain and inflammation, whereas MCR-4 ligands are useful for treating obesity. The binding characteristics and specificity of a given MC receptor ligand can be selected based on the particular disease or physiological effect that is desired to be altered.

Another assay useful for identifying or characterizing MC receptor ligands measures signaling of MC receptors. MC receptors are G protein-coupled receptors that couple to adenylate cyclase and produce cAMP. Therefore, measuring cAMP production in a cell expressing a MC receptor and treated with a MC receptor ligand can be used to assess the function of the MC receptor ligand in activating a MC receptor.

Ligands for MC-3 that can alter the activity of an MC-3 receptor can be useful for treating sexual dysfunction and other conditions or conditions associated with MC-3 such as inflammation. Other MC-3-associated conditions that can be treated with the MC-3 receptor ligands include disuse deconditioning; organ damage such as organ transplantation or ischemic injury; adverse reactions associated with cancer chemotherapy; diseases such as atherosclerosis that are mediated by free radicals and nitric oxide action; bacterial endotoxic sepsis and related shock; adult respiratory distress syndrome; and autoimmune or other patho-immunogenic diseases or reactions such as allergic reactions or anaphylaxis, rheumatoid arthritis, inflammatory bowel disease, ulcerative colitis, glomerulonephritis, systemic lupus erythematosus, transplant atherosclerosis and parasitic mediated immune dysfunctions such as Chagas's disease.

The invention further provides a method for treating an MC-3-associated condition in a subject. The term “MC-3-associated condition” includes any condition or condition mediated by MC-3 or can be affected by binding an MC-3 ligand. Such conditions include inflammation and sexual dysfunction.

The term “sexual dysfunction” herein means any condition that inhibits or impairs normal sexual function, including coitus. However, the term need not be limited to physiological conditions, but may include psychogenic conditions or perceived impairment without a formal diagnosis of pathology.

In males, sexual dysfunction includes erectile dysfunction. The term “erectile dysfunction” or “impotence” means herein the inability or impaired ability to attain or sustain an erection that would be of satisfactory rigidity for coitus. Sexual dysfunction in males can also include premature ejaculation and priapism, which is a condition of prolonged and sometimes painful erection unrelated to sexual activity, often associated with sickle-cell disease.

In females, sexual dysfunction includes sexual arousal disorder. The term “sexual arousal disorder” means herein a persistent or recurrent failure to attain or maintain the lubrication-swelling response of sexual excitement until completion of sexual activity. Sexual dysfunction in females can also include inhibited orgasm and dyspareunia, which is painful or difficult coitus. Sexual dysfunction can also be manifested as inhibited sexual desire or inhibited lordosis behavior in animals.

In addition, the ability of the compounds to inhibit bacterial growth, and therefore be useful to that infection, can be determined by methods well known in the art. Compounds of the present invention can be shown to have antimicrobial activity by the in vitro antimicrobial activity assay described below and, therefore, are useful as antimicrobial agents.

Moreover, an exemplary in vitro antimicrobial activity assay is described in Blondelle and Houghten, Biochemistry 30:4671-4678 (1991), which is incorporated herein by reference. In brief, Staphylococcus aureus ATCC 29213 (Rockville, Md.) is grown overnight at 37° C. in Mueller-Hinton broth, then re-inoculated and incubated at 37° C. to reach the exponential phase of bacterial growth (i.e., a final bacterial suspension containing 105 to 5×105 colony-forming units/ml). The concentration of cells is established by plating 100 μl of the culture solution using serial dilutions (e.g., 10-2, 10-3 and 10-4) onto solid agar plates. In 96-well tissue culture plates, compounds, individual or in mixtures, are added to the bacterial suspension at concentrations derived from serial two-fold dilutions ranging from 1500 to 2.9 μg/ml. The plates are incubated overnight at 37° C. and the growth determined at each concentration by OD620 nm. The IC50 (the concentration necessary to inhibit 50% of the growth of the bacteria) can then be calculated.

The competitive ELISA method which can be used here is a modification of the direct ELISA technique described previously in Appel et al., J. Immunol. 144:976-983 (1990), which is incorporated herein by reference. It differs only in the MAb addition step. Briefly, multi-well microplates are coated with the antigenic peptide (Ac-GASPYPNLSNQQT-NH2) at a concentration of 100 pmol/50 μl. After blocking, 25 μl of a 1.0 mg/ml solution of each mixture of a synthetic combinatorial library (or individual compound) is added, followed by MAb 125-10F3 (Appel et al., supra) (25 μl per well). The MAb is added at a fixed dilution in which the isothiourea in solution effectively competes for MAb binding with the antigenic peptide adsorbed to the plate. The remaining steps are the same as for direct ELISA. The concentration of compound necessary to inhibit 50% of the MAb binding to the control peptide on the plate (IC50) is determined by serial dilutions of the compound.

Alternative screening can be done with radio-receptor assays. The radio-receptor assay, can be selective for any one of the μ, κ, or δ opiate receptors. Compounds of the present invention can be useful in vitro for the diagnosis of relevant opioid receptor subtypes, such as κ, in the brain and other tissue samples. Similarly, the compounds can be used in vivo diagnostically to localize opioid receptor subtypes.

The radio-receptor assays are also an indication of the compounds' analgesic properties as described, for example, in Dooley et al., Proc. Natl. Acad. Sci., 90:10811-10815 (1993). For example, it can be envisioned that these compounds can be used for therapeutic purposes to block the peripheral effects of a centrally acting pain killer. For instance, morphine is a centrally acting pain killer. Morphine, however, has a number of deleterious effects in the periphery which are not required for the desired analgesic effects, such as constipation and pruritus (itching). While it is known that the many compounds do not readily cross the blood-brain barrier and, therefore, elicit no central effect, the subject compounds can have value in blocking the periphery effects of morphine, such as constipation and pruritus. Accordingly, the subject compounds can also be useful as drugs, namely as analgesics, or to treat pathologies associated with other compounds which interact with the opioid receptor system.

Additionally, such compounds can be tested in a a receptor assay. Ligands for the σ receptor can be useful as antipsychotic agents, as described in Abou-Gharbia et al., Annual Reports in Medicinal Chemistry, 28:1-10 (1993).

Radio-receptor assays can be performed with particulate membranes prepared using a modification of the method described in Pasternak et al., Mol. Pharmacol. 11:340-351 (1975), which is incorporated herein by reference. Rat brains frozen in liquid nitrogen can be obtained from Rockland (Gilbertsville, Pa.). The brains are thawed, the cerebella removed and the remaining tissue weighed. Each brain is individually homogenized in 40 ml Tris-HCl buffer (50 mM, pH 7.4, 4° C.) and centrifuged (Sorvall® RC5C SA-600: Du Pont, Wilmington, Del.) (16,000 rpm) for 10 minutes. The pellets are resuspended in fresh Tris-HCl buffer and incubated at 37° C. for 40 minutes. Following incubation, the suspensions are centrifuged as before, the resulting pellets resuspended in 100 volumes of Tris buffer and the suspensions combined. Membrane suspensions are prepared and used in the same day. Protein content of the crude homogenates generally range from 0.15-0.2 mg/ml as determined using the method described in Bradford, M. M., Anal. Biochem. 72:248-254 (1976), which is incorporated herein by reference.

Binding assays are carried out in polypropylene tubes, each tube containing 0.5 ml of membrane suspension. 8 nM of 3H-[D-Ala2,Me-Phe4,Gly-o15]enkephalin (DAMGO) (specific activity=36 Ci/mmol, 160,000 cpm per tube; which can be obtained from Multiple Peptide Systems, San Diego, Calif., through NIDA drug distribution program 271-90-7302) and 80 μg/ml of isothiourea, individual or as a mixture and Tris-HCl buffer in a total volume of 0.65 ml. Assay tubes are incubated for 60 mins. at 25° C. The reaction is terminated by filtration through GF-B filters on a Tomtec harvester (Orange, Conn.). The filters are subsequently washed with 6 ml of Tris-HCl buffer, 4° C. Bound radioactivity is counted on a Pharmacia Biotech Betaplate Liquid Scintillation Counter (Piscataway, N.J.) and expressed in cpm. To determine inter- and intra-assay variation, standard curves in which 3H-DAMGO is incubated in the presence of a range of concentrations of unlabeled DAMGO (0.13-3900 nM) are generally included in each plate of each assay (a 96-well format). Competitive inhibition assays are performed as above using serial dilutions of the isothioureas, individually or in mixtures. IC50 values (the concentration necessary to inhibit 50% of 3H-DAMGO binding) are then calculated. IC50 values of less than 1000 nM are indicative of highly active opioid compounds which bind to the μ receptor, with particularly active compounds having IC50 values of 100 nM or less and the most active compounds with values of less than 10 nM.

As opposed to this μ receptor selective assay, which can be carried out using 3H-DAMGO as radioligand, as described above, assays selective for κ receptors can be carried out using [3H]-U69,593 (3 nM, specific activity 62 Ci/mmol) as radioligand. Assays selective for δ opiate receptors can be carried out using tritiated DSLET ([D-Ser2, D-Leu5]-threonine-enkephalin) as radioligand. Assays selective for the σ opiate receptor can use radiolabeled pentazocine as ligand.

Screening of combinatorial libraries and compounds of the invention can be done with an anti-fungal assay. Compounds of the present invention can be useful for treating fungal infections.

Screening of combinatorial libraries and compounds of the invention also can be done with a calmodulin-dependent phosphodiesterase (CaMPDE) assay. Compounds of the present invention can be useful as calmodulin antagonists.

Calmodulin (CaM), which is the major intracellular calcium receptor, is involved in many processes that are crucial to cellular viability. In particular, calmodulin is implicated in calcium-stimulated cell proliferation. Calmodulin antagonists are, therefore, useful for treating conditions associated with increased cell proliferation, for example, cancer. In addition, calmodulin antagonists such as compounds of the subject invention are useful both in vitro and in vivo for identifying the role of calmodulin in other biological processes. The disadvantages of known antagonists such as trifluoperazine and N-(4-aminobutyl)-5-chloro-2-naphthalenesulfonamide (W13) include their non-specificity and toxicity. In contrast, advantages of the combinatorial libraries and compounds of the subject invention as calmodulin antagonists include their reduced flexibility and ability to generate broader conformational space of interactive residues as compared to their linear counterparts.

An example of an assay that identifies CaM antagonists is a CaMPDE assay. In brief, samples are mixed with 50 μl of assay buffer (360 mM Tris, 360 mM Imidazole, 45 mM Mg(CH3COO)2, pH 7.5) and 10 μl of CaCl2 (4.5 mM) to a final volume of 251 μl. 25 μl of calmodulin stock solution (Boehringer Mannheim; 0.01 μg/μl) is then added and the samples then sit at room temperature for 10 minutes. 14 μl of PDE (Sigma; 2 Units dissolved in 4 ml of water; stock concentration: 0.0005 Units/μl) is then added, followed by 50 μl of 5′-nucleotidase (Sigma; 100 Units dissolved in 10 ml of 10 mM Tris-HCl containing 0.5 mM Mg(CH3COO)2, pH 7.0; stock concentration: 10 Units/ml). The samples are then incubated for 10 minutes at 30° C. 50 μl of adenosine 3′,5′-cyclic monophosphate (cAMP) (20 mM in water at pH 7.0) is added, the samples incubated for 1 hour at 30° C. and then vortexed. 200 μl of trichloroacetic acid (TCA) (55% in water) is added to a 200 μl sample aliquot, which is then vortexed and centrifuged for 10 minutes. 80 μl of the resulting supernatants of each sample is transferred to a 96-well plate, with 2 wells each containing 80 μl of each sample. 80 μl of ammonium molybdate (1.1% in 1.1N H2SO4) is then added to all the wells, and the OD of each were determined at 730 nm, with the values later subtracted to the final OD reading. 16 μl of reducing agent (6 g sodium bisulfite, 0.6 g sodium sulfite and 125 mg of 1-amino-2-naphtol-4-sulfonic acid in 50 ml of water) is then added to one of each sample duplicate and 16 μl of water is added to the other duplicate. After sitting for 1 hour at room temperature, the OD of each well is determined at 730 nm. The percent inhibition of calmodulin activity is then calculated for each sample, using as 0% inhibition a control sample containing all reagents without any test samples and as 100% inhibition a control sample containing test samples and all reagents except calmodulin. In addition, the percent inhibition of phosphodiesterase activity was determined by following a similar protocol as the CaMPDE assay described above, except not adding calmodulin to the sample mixture and calculating the percent inhibition by using as 0% inhibition a control reagent without any test samples and as 100% inhibition a control sample containing test samples and all reagents except cAMP.

The following examples are provided to illustrate but not limit the present invention. The following abreviations have the corresponding meanings:

DMF: N,N-dimethylforamide;

HOBt: 1-hydroxybenzotriazole;

Boc: tert-butoxycarbonyl;

DIC: N,N′-diisopropylcarbodiimide;

TFA: trifluoroacetic acid;

DIEA: N,N-diisopropylethylamine;

DCM: dichloromethane;

RT: room temperature

MeOH: methanol

### EXAMPLE 1

### Synthetic Protocol

Step 1. General Procedure for Preparation of Br-Wang Resin 2

1.6 g of Wang resin 1 (1.28 mml/g) was placed in porous polypropylene packet (Tea-bag), 60 mm×50 mm, 65μ), sealed and transferred to a 125 ml PP bottle. A freshly prepared solution of PPh3Br2 (6.1 mmol, 3.0 eq, 0.15 M) in DCM (40 mL) was added to the packet. After shaking for 4-6 hours at room temperature, the packet was washed with DCM (5×80 ml) and diethyl ether (4×80 ml). The packet was dried overnight under vacuum to afford off-white resin 2.

Step 2. Preparation of Resin Bound Aldehydes 3.

The packet containing freshly prepared Br-Wang resin 2 was transferred to an appropriate glass bottle, to which an Aldehyde (20 mmol, 10 eq, 0.2 M), anhydrous DMA (100 ml) and KOtBu (20 mmol, 10 eq, 0.2 M) were added sequentially. After heating at 50° C. for 4 hours, the packet was washed alternatively with DMF (3×80 mL) and MeOH (2×80 ml) followed by DCM (2×80 ml) and MeOH (3×80 ml). The packet was air-dried overnight to afford off-white to pale brown resin 3.

Step 3. Preparation of Resin Bound Amines 4.

The packet of Aldehyde-Wang resin 3 was transferred to a 250 mL PP bottle, to which a solution of Sodium Triacetoxyborohydride (NaBHAc₃, 20 mmol, 10 eq, 0.25 M) in DMF with 1% Acetic Acid and an Amine (20 mmol, 10 eq, 0.25 M) were added sequentially. After shaking at room temperature overnight, the packet was washed several times with DMF with 5% DIEA (2×80 mL) and alternatively with MeOH (80 mL) for 3 cycles, followed by washes of DCM (2×80 mL) and MeOH (3×80 mL). The packet was air-dried overnight to afford a yellow to dark orange colored resins 4, depending on the amine used.

Step 4. Preparation of Resin Bound Bt-Amidine 5.

The tea bag containing the Amine 4 was transferred to a 250 ml PP bottle, to which a solution of Di(benzotriazole-1-yl)Methanimine (10 mmol, 5 eq, 0.25M) in THF. After shaking for six hours at room temperature, the tea bag was washed several times with THF (2×80 ml) followed by washes of DCM (80 ml) and MeOH (80 ml) alternatively for three cycles. The packet was air-dried overnight to afford a yellow to off white resin 5.

Step 5. Preparation of Resin bound N,N-substituted, S-Aryl Isothioureas 6.

The packet containing the Wang resin Benzotriazole Amidine 5 was transferred to a 250 ml PP bottle, to which a solution of Thiophenol (20 mmol, 10 eq, 0.25M) in THF (60 ml) was added. After shaking for 24 hour at room temperature, the tea bag was washed several times with THF (2×60 ml), followed by washes of DCM (60 ml) alternating with MeOH (60 ml) for three cycles. The packet was air-dried overnight to afford a yellow to off white colored resin 6.

Step 6. Preparation of Resin bound N,N,N′-substituted, S-Aryl Isothioureas 7.

The tea bag was cut open and the resin 6 was distributed equally into 40 wells of a microtiter plate. The walls of each well were washed with MeOH (1.0 mL per well) and the excess solvent was removed by aspiration. The plate was air-dried overnight to afford loose-resin (approx. 0.050 mmol) at the bottom of each well. A solution of an appropriate Isocyanate (0.5 mmol, 10 eq, 0.4 M) in THF (1 mL) was added to each well under a nitrogen atmosphere to exclude moisture. The plate was tightly capped, and shaken for 24 hours at RT. Each plate was washed alternatively with DMF (6×1 mL/well) and MeOH (8×1 mL/well), then the plate was air-dried overnight and under vacuum for 4 hours to afford resin 7.

Step 7. Cleavage from Linker and Extraction

To dry the microtiter plate containing the Isothiourea resin 6 or 7, 20% TFA/DCM (0.5 ml) was added to each well. The plate was capped and placed on a shaker at room temperature for 3 h, and the solvent removed in a centrifugal evaporator. Acetic Acid was added to extract the product from the resin, and the extract frozen and lyophilized to provide the title compounds 8 or 9. All of the final products were analyzed by HPLC/MS using ELSD detection to determine their purity.

### EXAMPLE 2

### Anti-microbial Screen

Streptococcus pyogenes (ATCC# 97-03 14289) was grown in Todd Hewitt Broth (THB) (Difco Laboratories #0492-17-6) overnight until reaching an optical density of (OD=0.636@570 nm) by reading 0.1 ml in a 96 well microtiter plate in a Molecular Devices Thermomax. This preparation was kept frozen as stocks in 30% v/v glycerol in 1.5 ml aliquots at −70 mC until use. Prior to experiments, 6 ml aliquots were thawed and diluted into 50 ml 2×THB. 60 ul of this dilution was added to 92 wells of microtiter plate. To three wells THB (200 ul) was added to serve as a blank and a sterility control. Test compounds in DMSO and appropriate concentrations of DMSO were added to Growth/Solvent Controls at 0 time. Plates were read at 0 time at 570 nm in the Molecular Devices plate reader to obtain compounds correction factors for insoluble or colored compounds. Plates were read again at 4 hours.

Percent inhibition is calculated with the following formula:

Color correct=O.D. 0 hr - Blank 0 hr)-(Solvent Control Ohr - Blank 0 hr)

$$\% Inhibition = 100 - \frac{O . D . test compound 4 hr - Blank 4 hr - color correct O . D . }{ growth \text{/} solvent control 4 hr - Blank 4 hr}$$

Under this formula, the most active compounds tested were as follows:

| | | | | | Raw | | | | | | | |
|---|---|---|---|---|---|---|---|---|---|---|---|---|
| Library | Cmpd | Lot | Plate | Well | Data | Assay Result | Assay | Conc | mg ml | LionID | | |
| 10700 | 447 | 1 | 10700-006 | G 07 | 0.077 | 99.46830266 | Spy4H | 0.1776 | TR1070000447 | 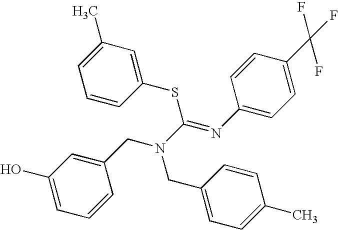 | C₃₀H₂₇F₃N₂OS | 520.616 |
| 10700 | 328 | 1 | 10700-005 | H 02 | 0.059 | 98.86920467 | Spy4H | 0.1776 | TR1070000328 | 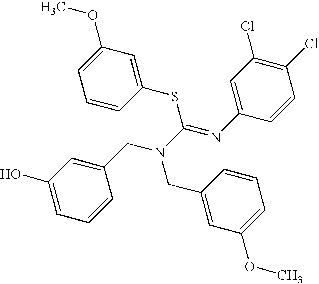 | C₂₉H₂₆Cl₂N₂O₃S | 553.507 |
| 10700 | 887 | 1 | 10700-012 | G 02 | 0.057 | 97.75474957 | Spy4H | 0.1776 | TR1070000887 | 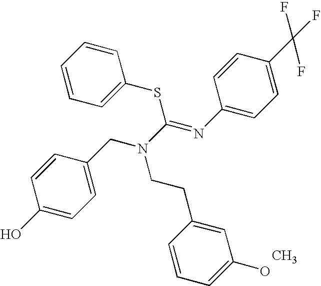 | C₃₀H₂₇F₃N₂O₂S | 536.615 |
| 10700 | 807 | 1 | 10700-011 | G 02 | 0.066 | 97.71573604 | Spy4H | 0.1776 | TR1070000807 | 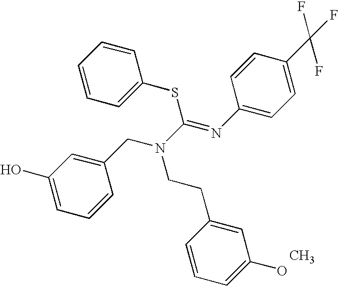 | C₃₀H₂₇F₃N₂O₂S | 536.615 |
| 10700 | 813 | 1 | 10700-011 | E 03 | 0.06 | 97.71573604 | Spy4H | 0.1776 | TR1070000813 | 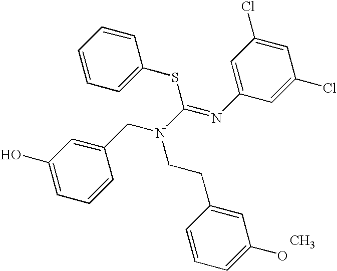 | C₂₉H₂₆Cl₂N₂O₂S | 537.508 |
| 10700 | 287 | 1 | 10700-004 | G 07 | 0.074 | 97.6825029 | Spy4H | 0.1776 | TR1070000287 | 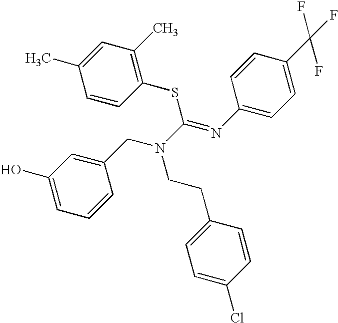 | C₃₁H₂₈ClF₃N₂OS | 569.088 |
| 10700 | 1143 | 1 | 10700-015 | G 04 | 0.065 | 97.0626918 | Spy4H | 0.1776 | TR1070001143 | 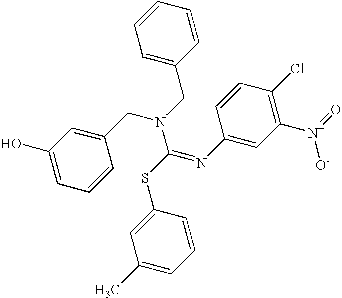 | C₂₈H₂₄ClN₃O₃S | 518.035 |
| 10700 | 527 | 1 | 10700-007 | G 07 | 0.061 | 96.43153527 | Spy4H | 0.1776 | TR1070000527 | 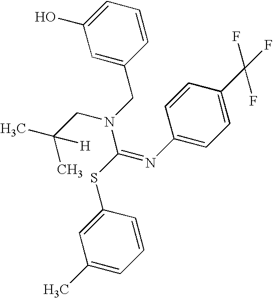 | C₂₆H₂₇F₃N₂OS | 472.572 |
| 10700 | 926 | 1 | 10700-012 | F 07 | 0.134 | 96.02763385 | Spy4H | 0.1776 | TR1070000926 | 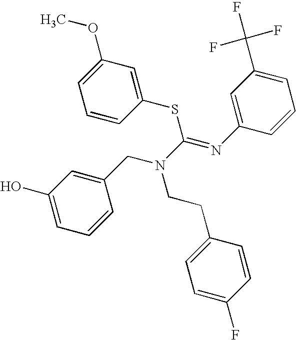 | C₃₀H₂₆F₄N₂O₂S | 554.605 |
| 10700 | 207 | 1 | 10700-003 | G 07 | 0.071 | 95.88777863 | Spy4H | 0.1776 | TR1070000207 | 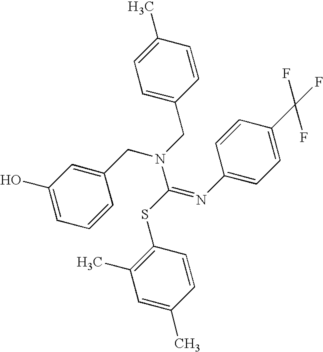 | C₃₁H₂₉F₃N₂OS | 534.643 |
| 10700 | 903 | 1 | 10700-012 | G 04 | 0.076 | 95.68221071 | Spy4H | 0.1776 | TR1070000903 | 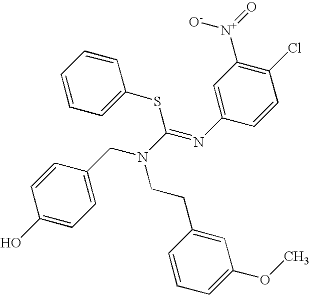 | C₂₉H₂₆ClN₃O₄S | 548.06 |
| 10700 | 509 | 1 | 10700-007 | E 05 | 0.162 | 94.77178423 | Spy4H | 0.1776 | TR1070000509 | 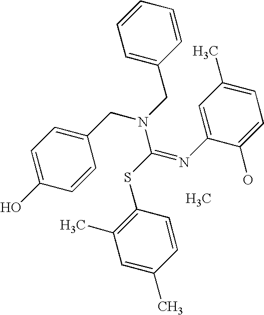 | C₃₁H₃₂N₂O₂S | 496.672 |
| 10700 | 133 | 1 | 10700-002 | E 08 | 0.082 | 94.27527406 | Spy4H | 0.1776 | TR1070000133 | 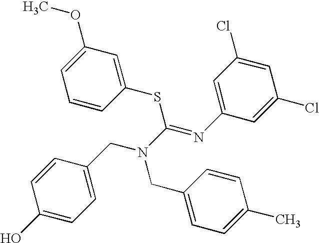 | C₂₉H₂₆Cl₂N₂O₂S | 537.508 |
| 10700 | 1167 | 1 | 10700-015 | G 07 | 0.116 | 94.25690487 | Spy4H | 0.1776 | TR1070001167 | 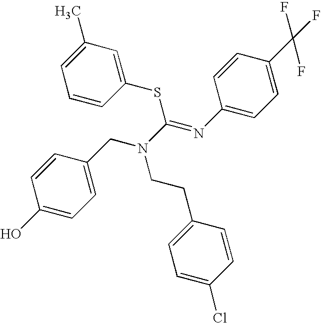 | C₃₀H₂₆ClF₃N₂OS | 555.061 |
| 10700 | 684 | 1 | 10700-009 | D 07 | 0.081 | 94.1203491 | Spy4H | 0.1776 | TR1070000684 | 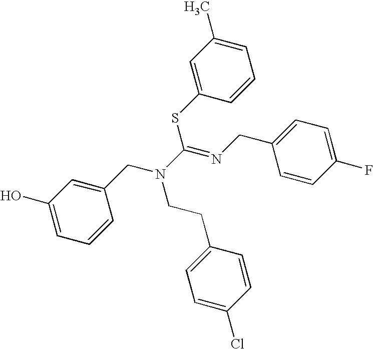 | C₃₀H₂₈ClFN₂OS | 519.081 |
| 10700 | 934 | 1 | 10700-012 | F 08 | 0.058 | 93.95509499 | Spy4H | 0.1776 | TR1070000934 | 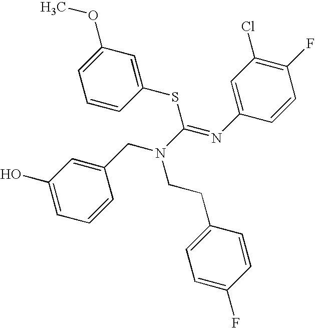 | C₂₉H₂₅ClF₂N₂O₂S | 539.044 |
| 10700 | 143 | 1 | 10700-002 | G 09 | 0.081 | 93.62565976 | Spy4H | 0.1776 | TR1070000143 | 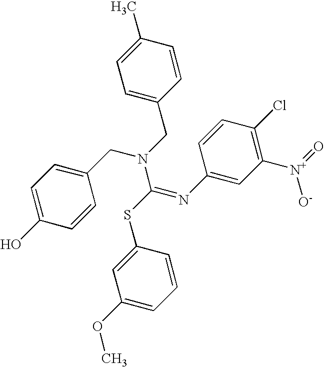 | C₂₉H₂₆ClN₃O₄S | 548.06 |
| 10700 | 847 | 1 | 10700-011 | G 07 | 0.089 | 90.27072758 | Spy4H | 0.1776 | TR1070000847 | 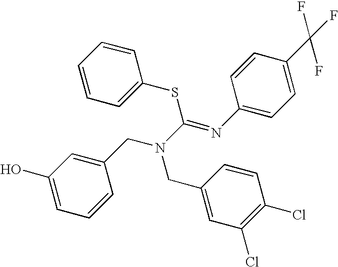 | C₂₈H₂₁Cl₂F₃N₂OS | 561.453 |
| 10700 | 1047 | 1 | 10700-014 | G 02 | 0.08 | 90.20025565 | Spy4H | 0.1776 | TR1070001047 | 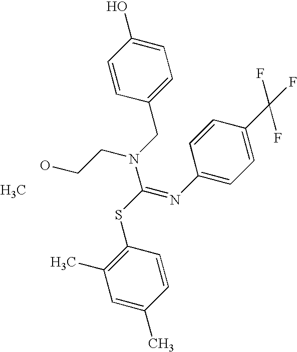 | C₂₆H₂₇F₃N₂O₂S | 488.571 |
| 10700 | 734 | 1 | 10700-010 | F 03 | 0.19 | 89.55353273 | Spy4H | 0.1776 | TR1070000734 | 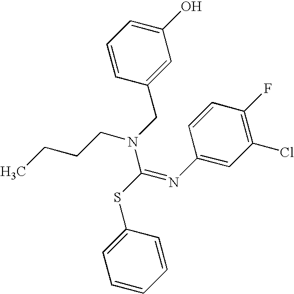 | C₂₄H₂₄ClFN₂OS | 442.984 |
| 10700 | 524 | 1 | 10700-007 | D 07 | 0.17 | 89.46058091 | Spy4H | 0.1776 | TR1070000524 | 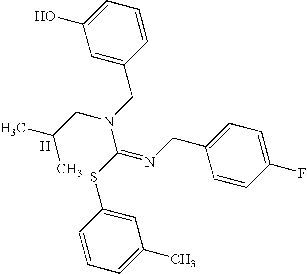 | C₂₆H₂₉FN₂OS | 436.592 |
| 10700 | 1127 | 1 | 10700-015 | G 02 | 0.088 | 88.645331 | Spy4H | 0.1776 | TR1070001127 | 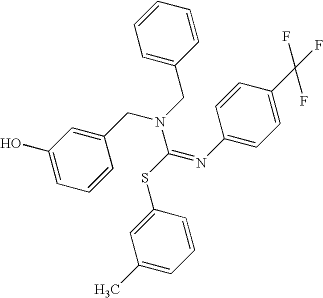 | C₂₉H₂₅F₃N₂OS | 506.59 |
| 10700 | 1265 | 1 | 10700-016 | A 10 | 0.13 | 88.33942439 | Spy4H | 0.1776 | TR1070001265 | 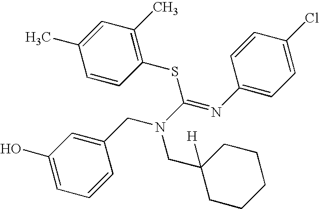 | C₂₉H₃₃ClN₂OS | 493.112 |
| 10700 | 676 | 1 | 10700-009 | D 06 | 0.087 | 87.50574185 | Spy4H | 0.1776 | TR1070000676 | 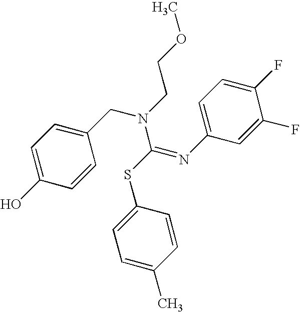 | C₂₄H₂₄F₂N₂O₂S | 442.528 |
| 10700 | 1093 | 1 | 10700-014 | E 08 | 0.124 | 87.13250959 | Spy4H | 0.1776 | TR1070001093 | 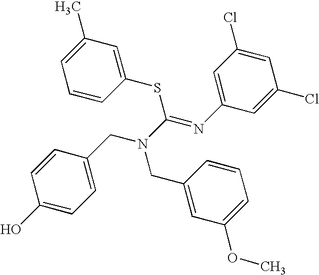 | C₂₉H₂₆Cl₂N₂O₂S | 537.508 |
| 10700 | 546 | 1 | 10700-007 | B 10 | 0.095 | 86.80497925 | Spy4H | 0.1776 | TR1070000546 | 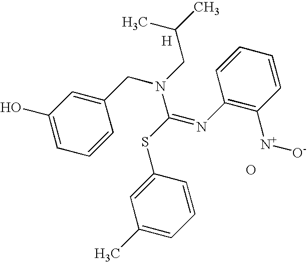 | C₂₅H₂₇N₃O₃S | 449.572 |
| 10700 | 463 | 1 | 10700-006 | G 09 | 0.102 | 86.70756646 | Spy4H | 0.1776 | TR1070000463 | 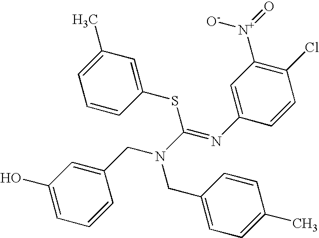 | C₂₉H₂₆ClN₃O₃S | 532.061 |
| 10700 | 73 | 1 | 10700-001 | A 11 | 0.097 | 85.78833693 | Spy4H | 0.1776 | TR1070000073 | 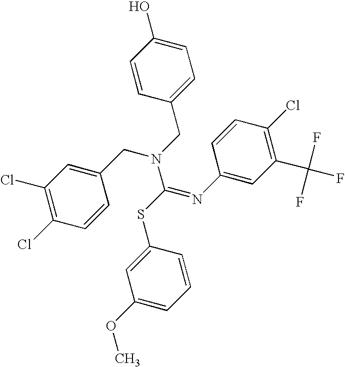 | C₂₉H₂₂Cl₃F₃N₂O₂S | 625.924 |
| 10700 | 47 | 1 | 10700-001 | G 07 | 0.097 | 85.78833693 | Spy4H | 0.1776 | TR1070000047 | 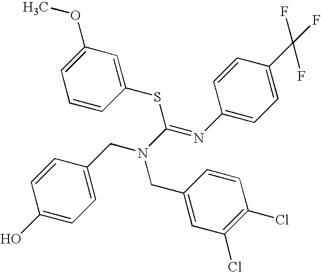 | C₂₉H₂₃Cl₂F₃N₂O₂S | 591.479 |
| 10700 | 1173 | 1 | 10700-015 | E 08 | 0.162 | 84.78737396 | Spy4H | 0.1776 | TR1070001173 | 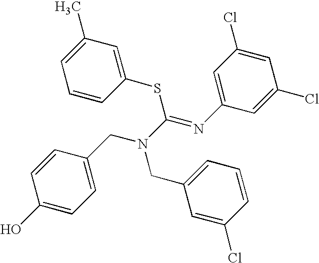 | C₂₉H₂₅Cl₃N₂OS | 555.955 |
| 10700 | 922 | 1 | 10700-012 | B 07 | 0.158 | 84.28324698 | Spy4H | 0.1776 | TR1070000922 | 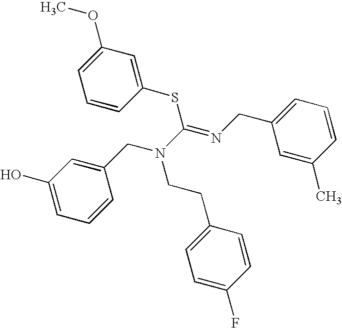 | C₃₁H₃₁FN₂O₂S | 514.662 |
| 10700 | 724 | 1 | 10700-010 | D 02 | 0.099 | 83.31166016 | Spy4H | 0.1776 | TR1070000724 | 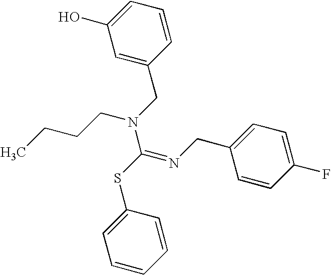 | C₂₅H₂₇FN₂OS | 422.565 |
| 10700 | 487 | 1 | 10700-007 | G 02 | 0.106 | 82.82157676 | Spy4H | 0.1776 | TR1070000487 | 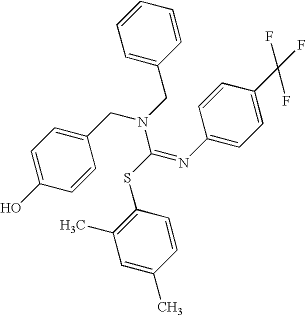 | C₃₀H₂₇F₃N₂OS | 520.616 |
| 10700 | 85 | 1 | 10700-002 | E 02 | 0.146 | 82.25740966 | Spy4H | 0.1776 | TR1070000085 | 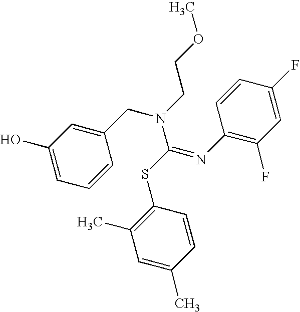 | C₂₅H₂₆F₂N₂O₂S | 456.554 |
| 10700 | 90 | 1 | 10700-002 | B 03 | 0.159 | 81.28298823 | Spy4H | 0.1776 | TR1070000090 | 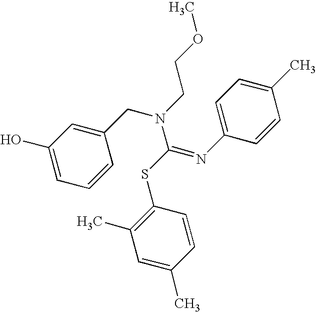 | C₂₆H₃₀N₂O₂S | 434.601 |
| 10700 | 844 | 1 | 10700-011 | D 07 | 0.121 | 80.79526227 | Spy4H | 0.1776 | TR1070000844 | 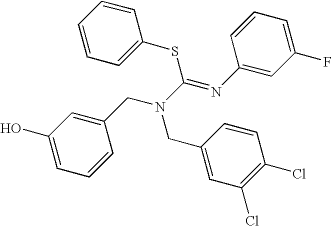 | C₂₈H₂₃Cl₂FN₂OS | 525.473 |
| 10700 | 653 | 1 | 10700-009 | E 03 | 0.113 | 80.15617823 | Spy4H | 0.1776 | TR1070000653 | 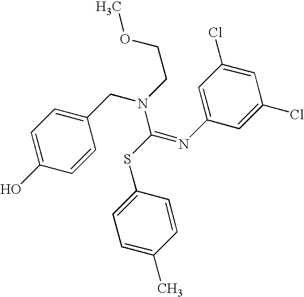 | C₂₄H₂₄Cl₂N₂O₂S | 475.438 |
| 10700 | 84 | 1 | 10700-002 | D 02 | 0.147 | 79.6589525 | Spy4H | 0.1776 | TR1070000084 | 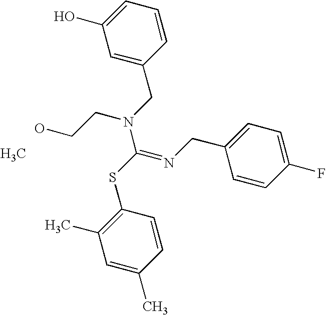 | C₂₆H₂₉FN₂O₂S | 452.591 |
| 10700 | 429 | 1 | 10700-006 | E 05 | 0.173 | 79.50920245 | Spy4H | 0.1776 | TR1070000429 | 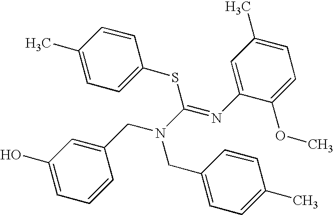 | C₃₁H₃₂N₂O₂S | 496.672 |
| 10700 | 893 | 1 | 10700-012 | E 03 | 0.116 | 79.10189983 | Spy4H | 0.1776 | TR1070000893 | 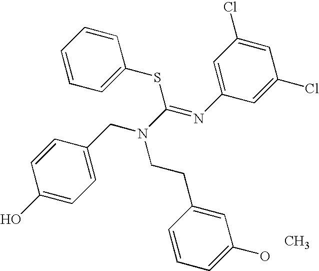 | C₂₉H₂₆Cl₂N₂O₂S | 537.508 |
| 10700 | 1149 | 1 | 10700-015 | E 05 | 0.203 | 76.01928979 | Spy4H | 0.1776 | TR1070001149 | 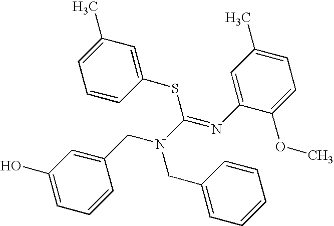 | C₃₀H₃₀N₂O₂S | 482.645 |
| 10700 | 107 | 1 | 10700-002 | C 05 | 0.176 | 75.76126675 | Spy4H | 0.1776 | TR1070000107 | 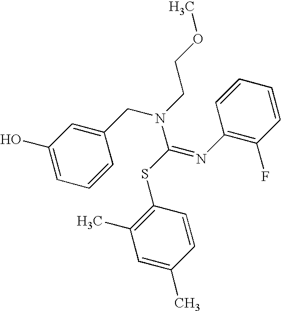 | C₂₅H₂₇FN₂O₂S | 438.564 |
| 10700 | 373 | 1 | 10700-005 | E 08 | 0.143 | 75.04711647 | Spy4H | 0.1776 | TR1070000373 | 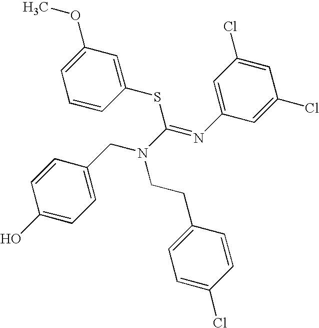 | C₂₉H₂₅Cl₃N₂O₂S | 571.953 |
| 10700 | 226 | 1 | 10700-003 | B 10 | 0.175 | 74.36587241 | Spy4H | 0.1776 | TR1070000226 | 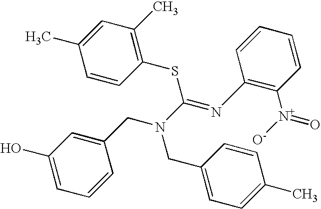 | C₃₀H₂₉N₃O₃S | 511.643 |
| 10700 | 1063 | 1 | 10700-014 | G 04 | 0.13 | 73.83894333 | Spy4H | 0.1776 | TR1070001063 | 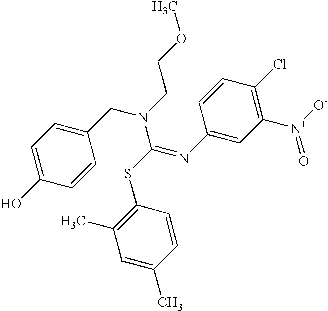 | C₂₅H₂₆ClN₃O₄S | 500.016 |
| 10700 | 673 | 1 | 10700-009 | A 06 | 0.126 | 72.80661461 | Spy4H | 0.1776 | TR1070000673 | 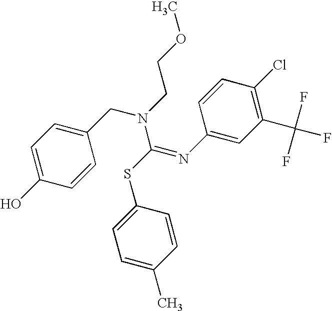 | C₂₅H₂₄ClF₃N₂O₂S | 508.99 |
| 10700 | 1164 | 1 | 10700-015 | D 07 | 0.137 | 72.16133275 | Spy4H | 0.1776 | TR1070001164 | 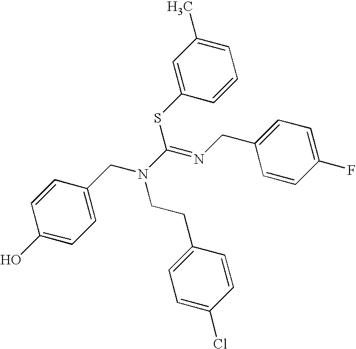 | C₃₀H₂₈ClFN₂OS | 519.081 |
| 10700 | 756 | 1 | 10700-010 | D 06 | 0.148 | 71.86822713 | Spy4H | 0.1776 | TR1070000756 | 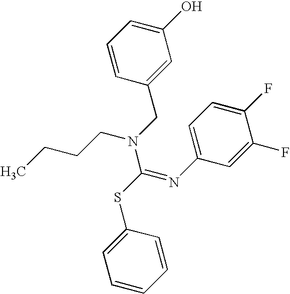 | C₂₄H₂₄F₂N₂OS | 426.529 |
| 10700 | 407 | 1 | 10700-006 | G 02 | 0.143 | 71.65644172 | Spy4H | 0.1776 | TR1070000407 | 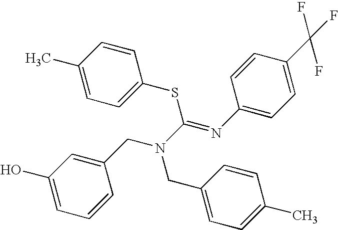 | C₃₀H₂₇F₃N₂OS | 520.616 |
| 10700 | 167 | 1 | 10700-003 | G 02 | 0.145 | 70.98385857 | Spy4H | 0.1776 | TR1070000167 | 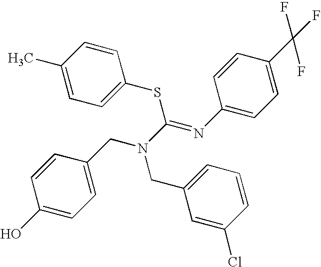 | C₂₉H₂₄ClF₃N₂OS | 541.035 |
| 10700 | 83 | 1 | 10700-002 | C 02 | 0.199 | 70.88915956 | Spy4H | 0.1776 | TR1070000083 | 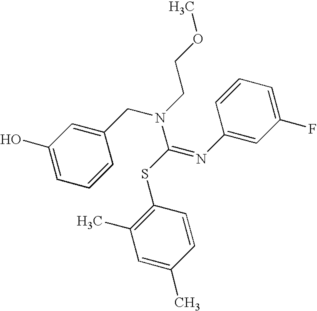 | C₂₅H₂₇FN₂O₂S | 438.564 |
| 10700 | 173 | 1 | 10700-003 | E 03 | 0.166 | 70.67640277 | Spy4H | 0.1776 | TR1070000173 | 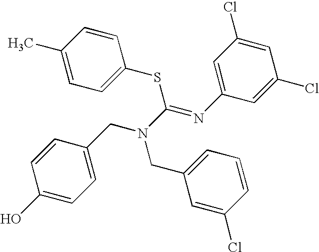 | C₂₈H₂₃Cl₃N₂OS | 541.928 |
| 10700 | 153 | 1 | 10700-002 | A 11 | 0.146 | 70.56435242 | Spy4H | 0.1776 | TR1070000153 | 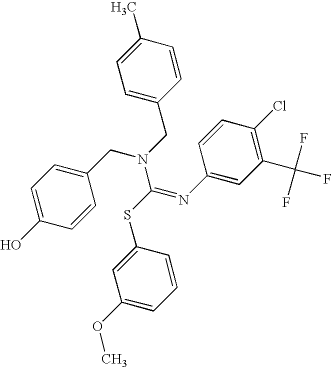 | C₃₀H₂₆ClF₃N₂O₂S | 571.06 |
| 10700 | 367 | 1 | 10700-005 | G 07 | 0.165 | 70.52393517 | Spy4H | 0.1776 | TR1070000367 | 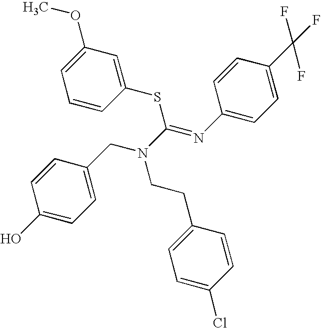 | C₃₀H₂₆ClF₃N₂O₂S | 571.06 |
| 10700 | 53 | 1 | 10700-001 | E 08 | 0.154 | 70.23758099 | Spy4H | 0.1776 | TR1070000053 | 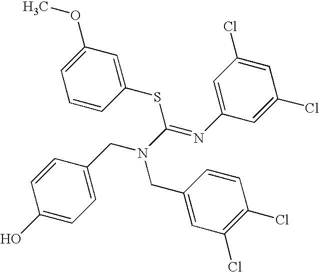 | C₂₈H₂₂Cl₄N₂O₂S | 592.372 |
| 10700 | 1133 | 1 | 10700-015 | E 03 | 0.138 | 70.05699255 | Spy4H | 0.1776 | TR1070001133 | 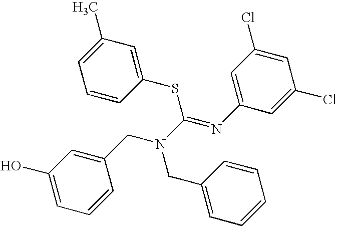 | C₂₈H₂₄Cl₂N₂OS | 507.483 |
| 10700 | 98 | 1 | 10700-002 | B 04 | 0.256 | 69.91473812 | Spy4H | 0.1776 | TR1070000098 | 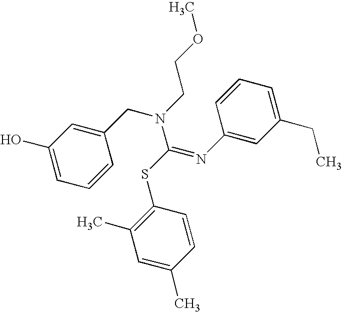 | C₂₇H₃₂N₂O₂S | 448.628 |
| 10700 | 466 | 1 | 10700-006 | B 10 | 0.171 | 69.69325153 | Spy4H | 0.1776 | TR1070000466 | 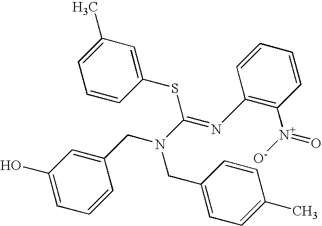 | C₂₉H₂₇N₃O₃S | 497.616 |
| 10700 | 204 | 1 | 10700-003 | D 07 | 0.178 | 69.13912375 | Spy4H | 0.1776 | TR1070000204 | 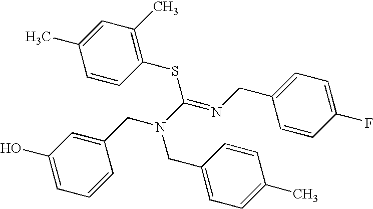 | C₃₁H₃₁FN₂OS | 498.663 |
| 10700 | 773 | 1 | 10700-010 | E 08 | 0.161 | 68.40052016 | Spy4H | 0.1776 | TR1070000773 | 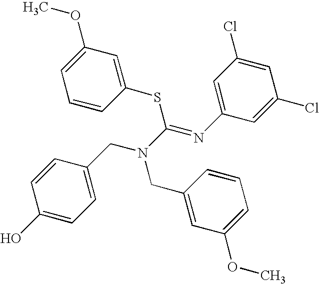 | C₂₉H₂₆Cl₂N₂O₃S | 553.507 |
| 10700 | 686 | 1 | 10700-009 | F 07 | 0.185 | 68.39687644 | Spy4H | 0.1776 | TR1070000686 | 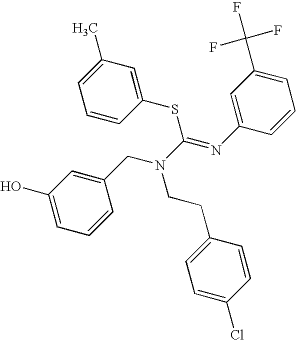 | C₃₀H₂₆ClF₃N₂OS | 555.061 |
| 10700 | 87 | 1 | 10700-002 | G 02 | 0.155 | 68.2907024 | Spy4H | 0.1776 | TR1070000087 | 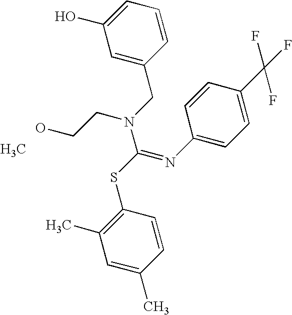 | C₂₆H₂₇F₃N₂O₂S | 488.571 |
| 10700 | 1241 | 1 | 10700-016 | A 07 | 0.167 | 68.19323892 | Spy4H | 0.1776 | TR1070001241 | 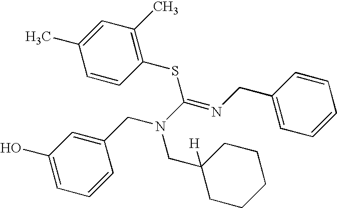 | C₃₀H₃₆N₂OS | 472.693 |
| 10700 | 1082 | 1 | 10700-014 | B 07 | 0.252 | 68.04431189 | Spy4H | 0.1776 | TR1070001082 | 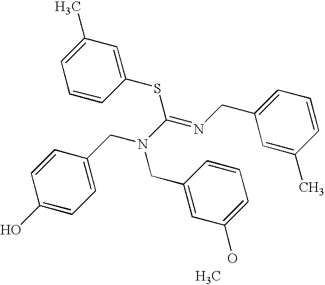 | C₁₃H₃₂N₂O₂S | 496.672 |
| 10700 | 44 | 1 | 10700-001 | D 07 | 0.176 | 67.47300216 | Spy4H | 0.1776 | TR1070000044 | 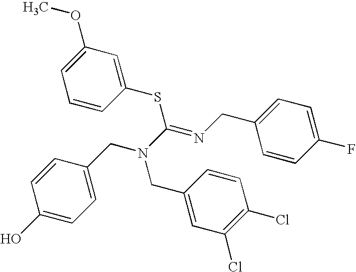 | C₂₉H₂₅Cl₂FN₂O₂S | 555.498 |
| 10700 | 426 | 1 | 10700-006 | B 05 | 0.186 | 67.40286299 | Spy4H | 0.1776 | TR1070000426 | 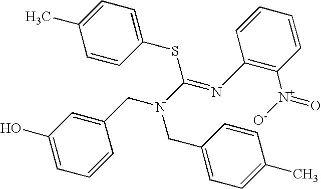 | C₂₉H₂₇N₃O₃S | 497.616 |
| 10700 | 1262 | 1 | 10700-016 | F 09 | 0.166 | 67.23389676 | Spy4H | 0.1776 | TR1070001262 | 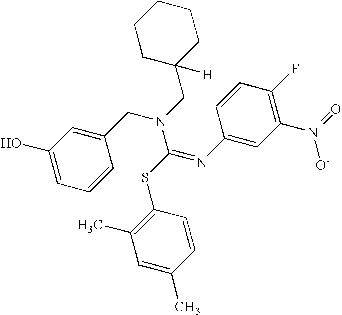 | C₂₉H₃₂FN₃O₃S | 521.654 |
| 10700 | 333 | 1 | 10700-005 | E 03 | 0.169 | 67.20693554 | Spy4H | 0.1776 | TR1070000333 | 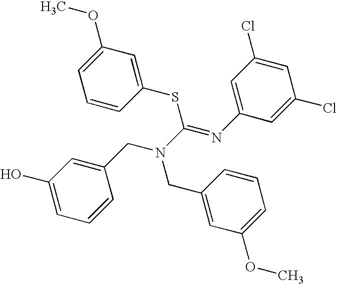 | C₂₉H₂₆Cl₂N₂O₃S | 553.507 |
| 10700 | 364 | 1 | 10700-005 | D 07 | 0.167 | 66.90539012 | Spy4H | 0.1776 | TR1070000364 | 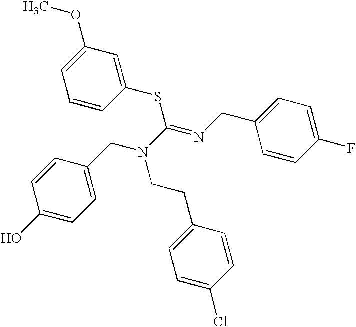 | C₃₀H₂₈ClFN₂O₂S | 535.08 |
| 10700 | 63 | 1 | 10700-001 | G 09 | 0.163 | 66.78185745 | Spy4H | 0.1776 | TR1070000063 | 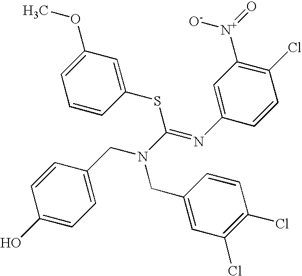 | C₂₈H₂₂Cl₃N₃O₄S | 602.924 |
| 10700 | 253 | 1 | 10700-004 | E 03 | 0.168 | 66.47354191 | Spy4H | 0.1776 | TR1070000253 | 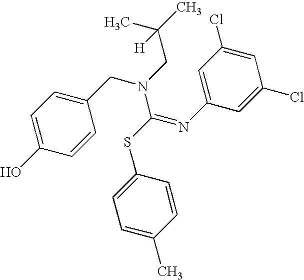 | C₂₅H₂₆Cl₂N₂OS | 473.465 |
| 10700 | 193 | 1 | 10700-003 | A 06 | 0.168 | 66.37202152 | Spy4H | 0.1776 | TR1070000193 | 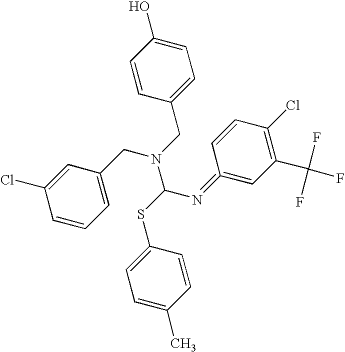 | C₂₉H₂₃Cl₂F₃N₂OS | 575.48 |
| 10700 | 935 | 1 | 10700-012 | G 08 | 0.116 | 66.32124352 | Spy4H | 0.1776 | TR1070000935 | 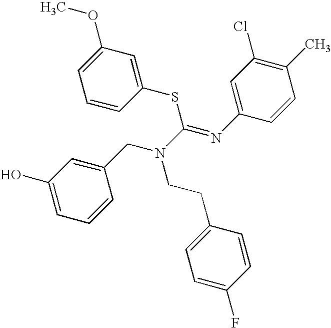 | C₃₀H₂₈ClFN₂O₂S | 535.08 |
| 10700 | 506 | 1 | 10700-007 | B 05 | 0.177 | 66.22406639 | Spy4H | 0.1776 | TR1070000506 | 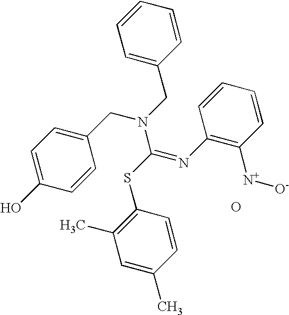 | C₂₉H₂₇N₃O₃S | 497.616 |
| 10700 | 1053 | 1 | 10700-014 | E 03 | 0.161 | 65.99914785 | Spy4H | 0.1776 | TR1070001053 | 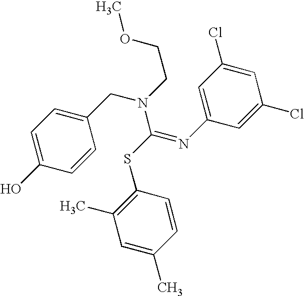 | C₂₅H₂₆Cl₂N₂O₂S | 489.464 |
| 10700 | 753 | 1 | 10700-010 | A 06 | 0.151 | 65.97312527 | Spy4H | 0.1776 | TR1070000753 | 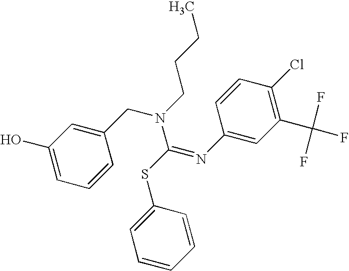 | C₂₅H₂₄ClF₃N₂OS | 492.991 |
| 10700 | 229 | 1 | 10700-003 | E 10 | 0.215 | 65.75710992 | Spy4H | 0.1776 | TR1070000229 | 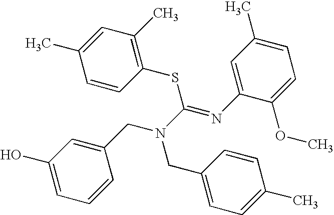 | C₃₂H₃₄N₂O₂S | 510.699 |
| 10700 | 13 | 1 | 10700-001 | E 03 | 0.162 | 65.74514039 | Spy4H | 0.1776 | TR1070000013 | 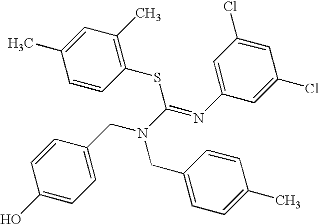 | C₃₀H₂₈Cl₂N₂OS | 535.536 |
| 10700 | 644 | 1 | 10700-009 | D 02 | 0.147 | 65.45705099 | Spy4H | 0.1776 | TR1070000644 | 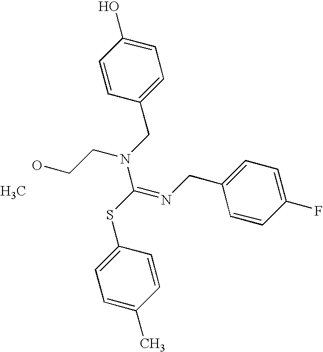 | C₂₅H₂₇FN₂O₂S | 438.564 |
| 10700 | 183 | 1 | 10700-003 | G 04 | 0.196 | 64.83474251 | Spy4H | 0.1776 | TR1070000183 | 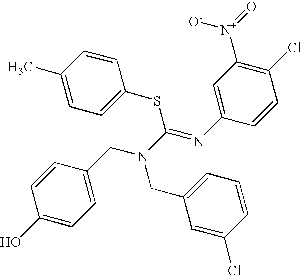 | C₂₈H₂₃Cl₂N₃O₃S | 552.48 |
| 10700 | 273 | 1 | 10700-004 | A 06 | 0.167 | 64.61954423 | Spy4H | 0.1776 | TR1070000273 | 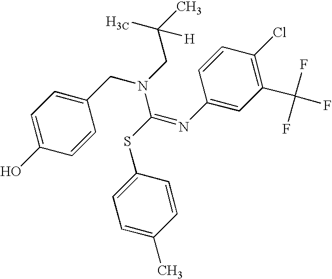 | C₂₆H₂₆ClF₃N₂OS | 507.017 |
| 10700 | 1113 | 1 | 10700-014 | A 11 | 0.155 | 64.29484448 | Spy4H | 0.1776 | TR1070001113 | 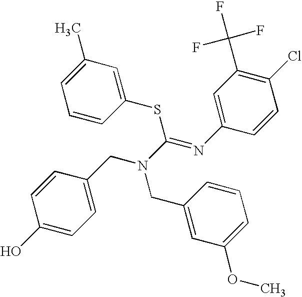 | C₃₀H₂₆ClF₃N₂O₂S | 571.06 |
| 10700 | 743 | 1 | 10700-010 | G 04 | 0.195 | 64.23927178 | Spy4H | 0.1776 | TR1070000743 | 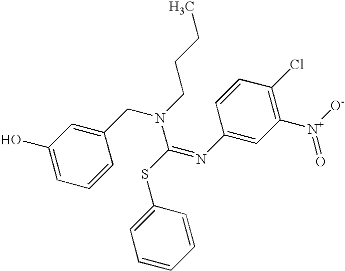 | C₂₄H₂₄ClN₃O₃S | 469.991 |
| 10700 | 284 | 1 | 10700-004 | D 07 | 0.196 | 64.001545 | Spy4H | 0.1776 | TR1070000284 | 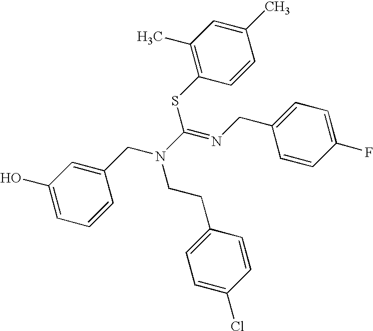 | C₃₁H₃₀ClFN₂OS | 533.108 |
| 10700 | 923 | 1 | 10700-012 | C 07 | 0.141 | 63.90328152 | Spy4H | 0.1776 | TR1070000923 | 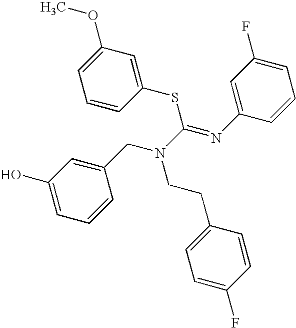 | C₂₉H₂₆F₂N₂O₂S | 504.598 |
| 10700 | 7 | 1 | 10700-001 | G 02 | 0.168 | 63.67170626 | Spy4H | 0.1776 | TR1070000007 | 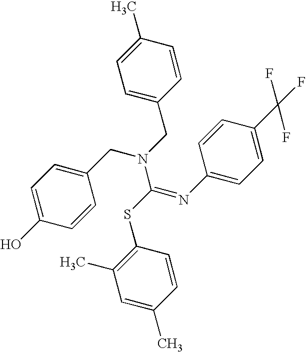 | C₃₁H₂₉F₃N₂OS | 534.643 |
| 10700 | 706 | 1 | 10700-009 | B 10 | 0.183 | 63.61966008 | Spy4H | 0.1776 | TR1070000706 | 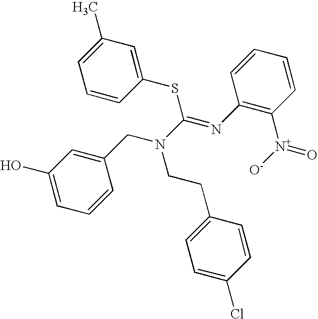 | C₂₉H₂₆ClN₃O₃S | 532.061 |
| 10700 | 733 | 1 | 10700-010 | E 03 | 0.183 | 63.54573039 | Spy4H | 0.1776 | TR1070000733 | 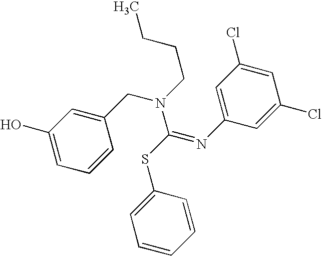 | C₂₄H₂₄Cl₂N₂OS | 459.439 |
| 10700 | 549 | 1 | 10700-007 | E 10 | 0.167 | 62.90456432 | Spy4H | 0.1776 | TR1070000549 | 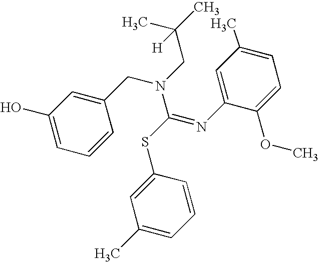 | C₂₇H₃₂N₂O₂S | 448.628 |
| 10700 | 1269 | 1 | 10700-016 | E 10 | 0.282 | 62.75696665 | Spy4H | 0.1776 | TR1070001269 | 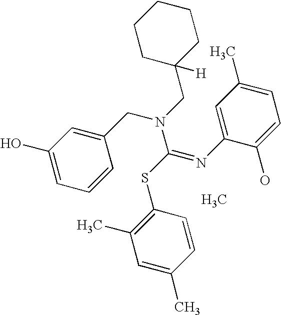 | C₃₁H₃₈N₂O₂S | 502.719 |
| 10700 | 1183 | 1 | 10700-015 | G 09 | 0.172 | 62.69180184 | Spy4H | 0.1776 | TR1070001183 | 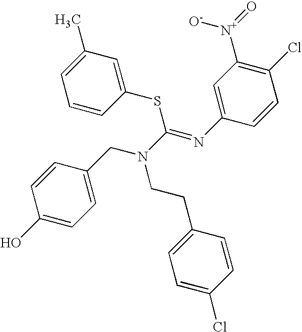 | C₂₉H₂₅Cl₂N₃O₃S | 566.506 |
| 10700 | 1153 | 1 | 10700-015 | A 06 | 0.158 | 62.69180184 | Spy4H | 0.1776 | TR1070001153 | 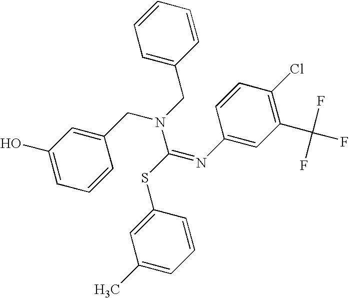 | C₂₉H₂₄ClF₃N₂OS | 541.035 |
| 10700 | 694 | 1 | 10700-009 | F 08 | 0.169 | 62.51722554 | Spy4H | 0.1776 | TR1070000694 | 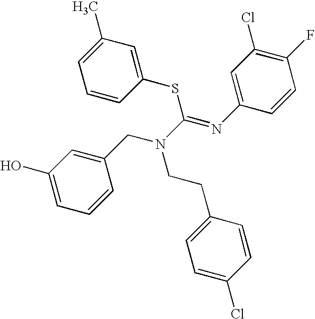 | C₂₉H₂₅Cl₂FN₂OS | 539.5 |
| 10700 | 869 | 1 | 10700-011 | E 10 | 0.183 | 62.18274112 | Spy4H | 0.1776 | TR1070000869 | 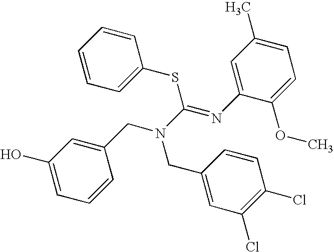 | C₂₉H₂₆Cl₂N₂O₂S | 537.508 |
| 10700 | 276 | 1 | 10700-004 | D 06 | 0.196 | 62.14754732 | Spy4H | 0.1776 | TR1070000276 | 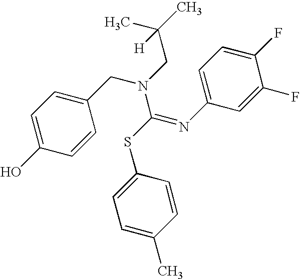 | C₂₅H₂₆F₂N₂OS | 440.555 |
| 10700 | 1254 | 1 | 10700-016 | F 08 | 0.18 | 62.11740521 | Spy4H | 0.1776 | TR1070001254 | 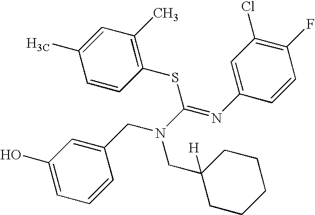 | C₂₉H₃₂ClFN₂OS | 511.102 |
| 10700 | 1266 | 1 | 10700-016 | B 10 | 0.229 | 61.79762449 | Spy4H | 0.1776 | TR1070001266 | 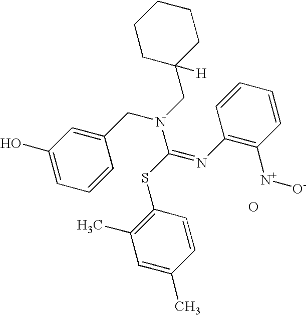 | C₂₉H₃₃N₃O₃S | 503.664 |
| 10700 | 189 | 1 | 10700-003 | E 05 | 0.215 | 61.76018447 | Spy4H | 0.1776 | TR1070000189 | 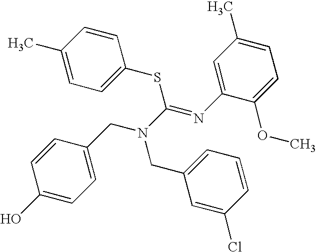 | C₃₀H₂₉ClN₂O₂S | 517.09 |
| 10700 | 1233 | 1 | 10700-016 | A 06 | 0.19 | 61.47784376 | Spy4H | 0.1776 | TR1070001233 | 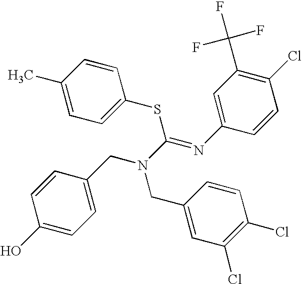 | C₂₉H₂₂Cl₃F₃N₂OS | 609.925 |
| 10700 | 127 | 1 | 10700-002 | G 07 | 0.176 | 61.46975233 | Spy4H | 0.1776 | TR1070000127 | 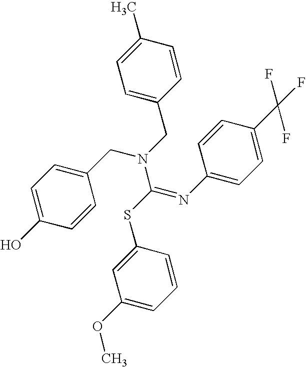 | C₃₀H₂₇F₃N₂O₂S | 536.615 |
| 10700 | 23 | 1 | 10700-001 | G 04 | 0.175 | 61.25269978 | Spy4H | 0.1776 | TR1070000023 | 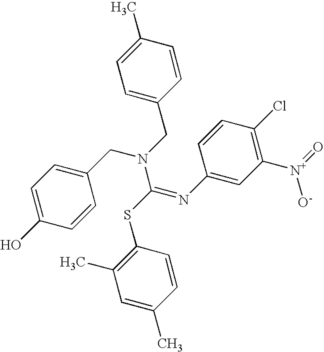 | C₃₀H₂₈ClN₃O₃S | 546.088 |
| 10700 | 866 | 1 | 10700-011 | B 10 | 0.184 | 60.82910321 | Spy4H | 0.1776 | TR1070000866 | 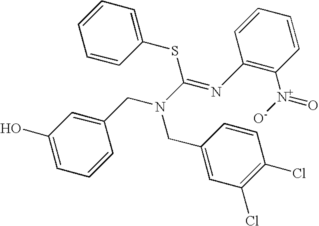 | C₂₇H₂₁Cl₂N₃O₃S | 538.453 |
| 10700 | 46 | 1 | 10700-001 | F 07 | 0.179 | 60.56155508 | Spy4H | 0.1776 | TR1070000046 | 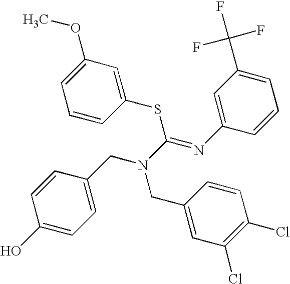 | C₂₉H₂₃Cl₂F₃N₂O₂S | 591.479 |
| 10700 | 453 | 1 | 10700-006 | E 08 | 0.182 | 60.53169734 | Spy4H | 0.1776 | TR1070000453 | 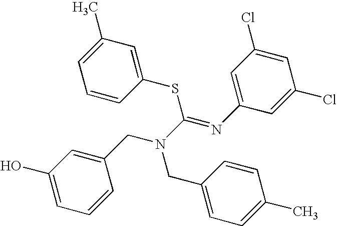 | C₂₉H₂₆Cl₂N₂OS | 521.509 |
| 10700 | 233 | 1 | 10700-003 | A 11 | 0.186 | 60.53036126 | Spy4H | 0.1776 | TR1070000233 | 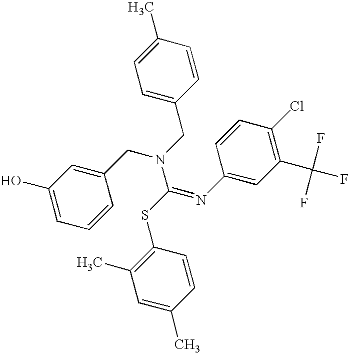 | C₃₁H₂₈ClF₃N₂OS | 569.088 |
| 10700 | 247 | 1 | 10700-004 | G 02 | 0.188 | 60.29354963 | Spy4H | 0.1776 | TR1070000247 | 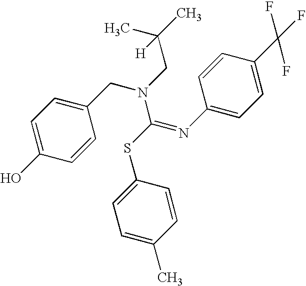 | C₂₆H₂₇F₃N₂OS | 472.572 |
| 10700 | 853 | 1 | 10700-011 | E 08 | 0.23 | 60.15228426 | Spy4H | 0.1776 | TR1070000853 | 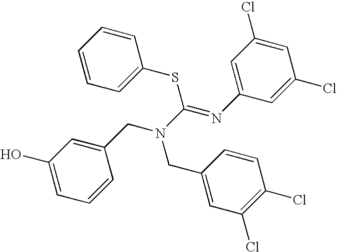 | C₂₇H₂₀Cl₄N₂OS | 562.346 |
| 10700 | 113 | 1 | 10700-002 | A 06 | 0.179 | 59.84571661 | Spy4H | 0.1776 | TR1070000113 | 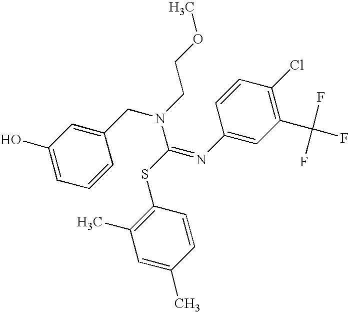 | C₂₆H₂₆ClF₃N₂O₂S | 523.016 |
| 10700 | 92 | 1 | 10700-002 | D 03 | 0.224 | 59.84571661 | Spy4H | 0.1776 | TR1070000092 | 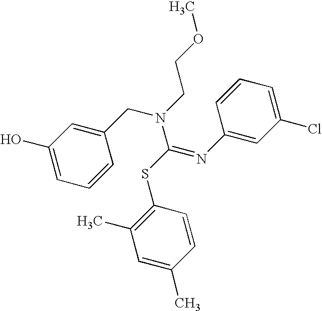 | C₂₅H₂₇ClN₂O₂S | 455.019 |
| 10700 | 933 | 1 | 10700-012 | E 08 | 0.169 | 59.7582038 | Spy4H | 0.1776 | TR1070000933 | 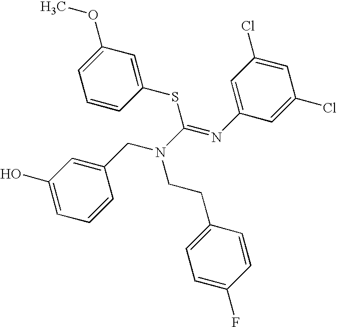 | C₂₉H₂₅Cl₂FN₂O₂S | 555.498 |
| 10700 | 1156 | 1 | 10700-015 | D 06 | 0.173 | 59.53529154 | Spy4H | 0.1776 | TR1070001156 | 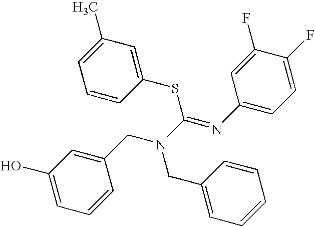 | C₂₈H₂₄F₂N₂OS | 474.573 |
| 10700 | 1103 | 1 | 10700-014 | G 09 | 0.207 | 59.52279506 | Spy4H | 0.1776 | TR1070001103 | 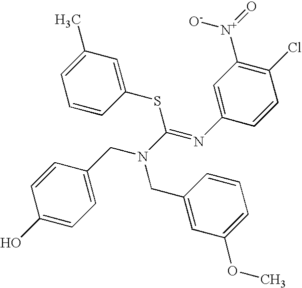 | C₂₉H₂₆ClN₃O₄S | 548.06 |
| 10700 | 353 | 1 | 10700-005 | A 06 | 0.188 | 59.0652092 | Spy4H | 0.1776 | TR1070000353 | 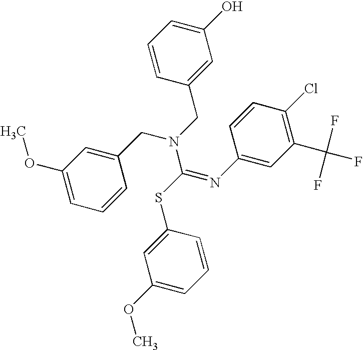 | C₂₉H₂₆ClF₃N₃O₄S | 587.059 |
| 10700 | 103 | 1 | 10700-002 | G 04 | 0.213 | 58.87129517 | Spy4H | 0.1776 | TR1070000103 | 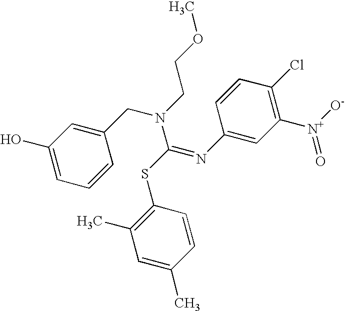 | C₂₅H₂₆ClN₃O₄S | 500.016 |
| 10700 | 26 | 1 | 10700-001 | B 05 | 0.214 | 58.1425486 | Spy4H | 0.1776 | TR1070000026 | 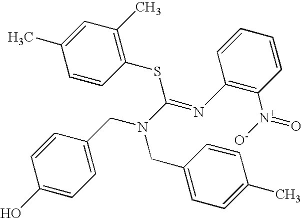 | C₃₀H₂₉N₃O₃S | 511.643 |
| 10700 | 1109 | 1 | 10700-014 | E 10 | 0.224 | 57.81849169 | Spy4H | 0.1776 | TR1070001109 | 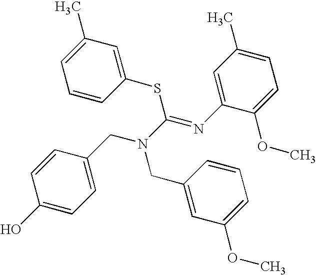 | C₃₁H₃₂N₂O₃S | 512.671 |
| 10700 | 33 | 1 | 10700-001 | A 06 | 0.187 | 57.79697624 | Spy4H | 0.1776 | TR1070000033 | 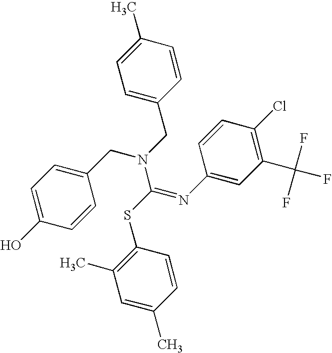 | C₃₁H₂₈ClF₃N₂OS | 569.088 |
| 10700 | 814 | 1 | 10700-011 | F 03 | 0.224 | 57.78341794 | Spy4H | 0.1776 | TR1070000814 | 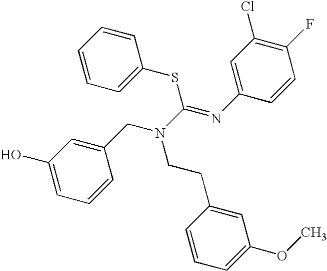 | C₂₉H₂₆ClFN₂O₂S | 521.053 |
| 10700 | 894 | 1 | 10700-012 | F 03 | 0.177 | 57.68566494 | Spy4H | 0.1776 | TR1070000894 | 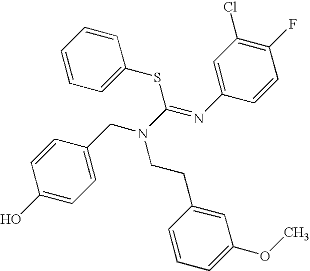 | C₂₉H₂₆ClFN₂O₂S | 521.053 |
| 10700 | 413 | 1 | 10700-006 | E 03 | 0.195 | 57.58691207 | Spy4H | 0.1776 | TR1070000413 | 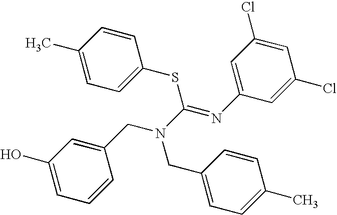 | C₂₉H₂₆Cl₂N₂OS | 521.509 |
| 10700 | 473 | 1 | 10700-006 | A 11 | 0.184 | 57.58691207 | Spy4H | 0.1776 | TR1070000473 | 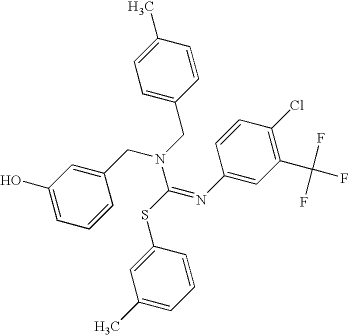 | C₃₀H₂₆ClF₃N₂OS | 555.061 |
| 10700 | 8 | 1 | 10700-001 | H 02 | 0.194 | 57.45140389 | Spy4H | 0.1776 | TR1070000008 | 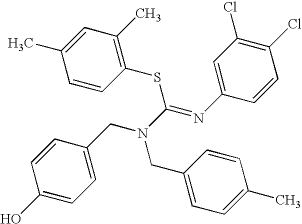 | C₃₀H₂₈Cl₂N₂OS | 535.536 |
| 10700 | 1260 | 1 | 10700-016 | D 09 | 0.199 | 57.32069438 | Spy4H | 0.1776 | TR1070001260 | 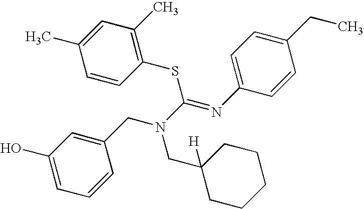 | C₃₁H₃₈N₂OS | 486.72 |
| 10700 | 1276 | 1 | 10700-016 | D 11 | 0.213 | 57.32069438 | Spy4H | 0.1776 | TR1070001276 | 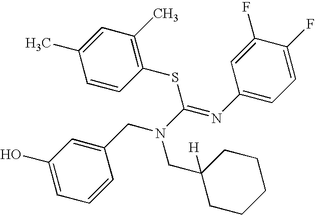 | C₂₉H₃₂F₂N₂OS | 494.647 |
| 10700 | 503 | 1 | 10700-007 | G 04 | 0.204 | 57.26141079 | Spy4H | 0.1776 | TR1070000503 | 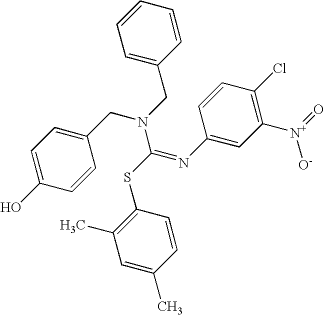 | C₂₉H₂₆ClN₃O₃S | 532.061 |
| 10700 | 423 | 1 | 10700-006 | G 04 | 0.192 | 57.2597137 | Spy4H | 0.1776 | TR1070000423 | 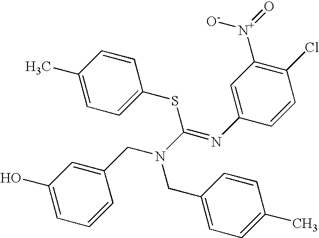 | C₂₉H₂₆ClN₃O₃S | 532.061 |
| 10700 | 213 | 1 | 10700-003 | E 08 | 0.216 | 57.14834743 | Spy4H | 0.1776 | TR1070000213 | 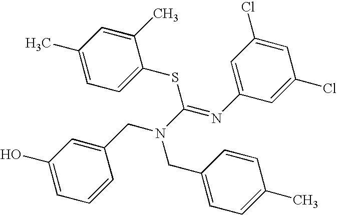 | C₃₀H₂₈Cl₂N₂OS | 535.536 |
| 10700 | 829 | 1 | 10700-011 | E 05 | 0.23 | 57.10659898 | Spy4H | 0.1776 | TR1070000829 | 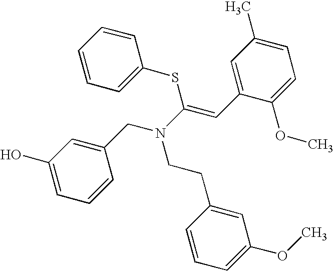 | C₃₁H₃₂N₂O₃S | 512.671 |
| 10700 | 36 | 1 | 10700-001 | D 06 | 0.182 | 56.76025918 | Spy4H | 0.1776 | TR1070000036 | 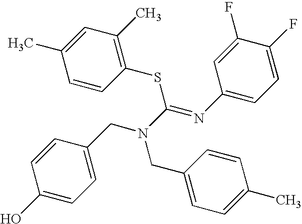 | C₃₀H₂₈F₂N₂OS | 502.626 |
| 10700 | 1263 | 1 | 10700-016 | G 09 | 0.225 | 56.68113294 | Spy4H | 0.1776 | TR1070001263 | 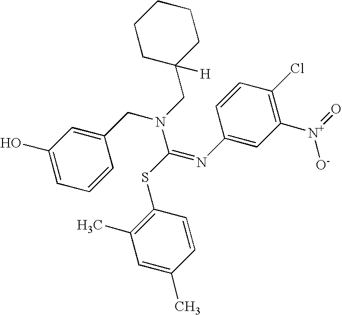 | C₂₉H₃₂ClN₃O₃S | 538.109 |
| 10700 | 327 | 1 | 10700-005 | G 02 | 0.196 | 56.65284583 | Spy4H | 0.1776 | TR1070000327 | 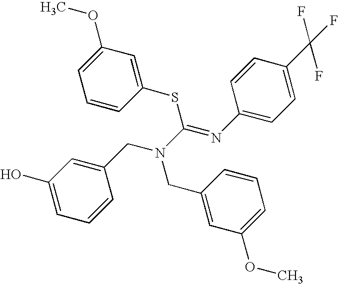 | C₃₀H₂₇F₃N₂O₃S | 552.614 |
| 10700 | 383 | 1 | 10700-005 | G 09 | 0.231 | 56.65284583 | Spy4H | 0.1776 | TR1070000383 | 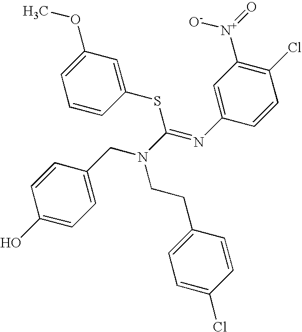 | C₂₉H₂₅Cl₂N₃O₄S | 582.505 |
| 10700 | 444 | 1 | 10700-006 | D 07 | 0.191 | 56.60531697 | Spy4H | 0.1776 | TR1070000444 | 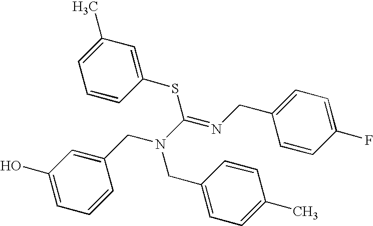 | C₃₀H₂₉FN₂OS | 484.636 |
| 10700 | 294 | 1 | 10700-004 | F 08 | 0.193 | 56.58555427 | Spy4H | 0.1776 | TR1070000294 | 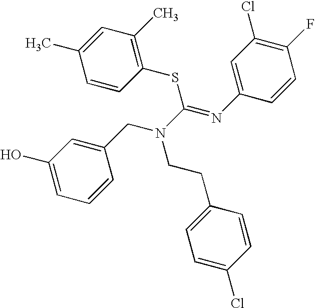 | C₃₀H₂₇Cl₂FN₂OS | 553.526 |
| 10700 | 186 | 1 | 10700-003 | B 05 | 0.228 | 56.53343582 | Spy4H | 0.1776 | TR1070000186 | 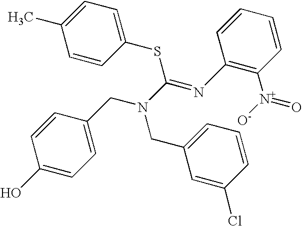 | C₂₈H₂₄ClN₃O₃S | 518.035 |
| 10700 | 1146 | 1 | 10700-015 | B 05 | 0.185 | 56.37878124 | Spy4H | 0.1776 | TR1070001146 | 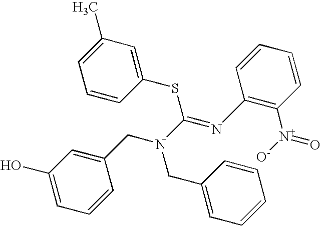 | C₂₈H₂₅N₃O₃S | 483.59 |
| 10700 | 936 | 1 | 10700-012 | H 08 | 0.136 | 56.30397237 | Spy4H | 0.1776 | TR1070000936 | 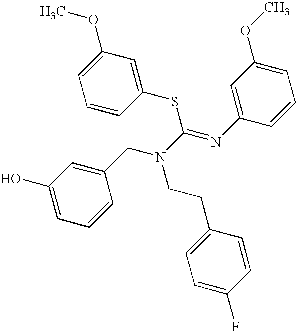 | C₃₀H₂₉FN₂O₃S | 516.634 |
| 10700 | 109 | 1 | 10700-002 | E 05 | 0.213 | 56.272838 | Spy4H | 0.1776 | TR1070000109 | 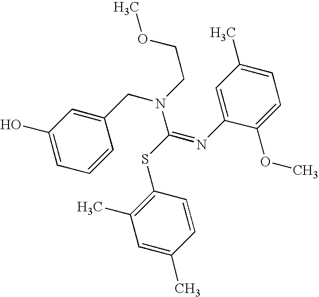 | C₂₇H₃₂N₂O₃S | 464.627 |
| 10700 | 727 | 1 | 10700-010 | G 02 | 0.178 | 56.26354573 | Spy4H | 0.1776 | TR1070000727 | 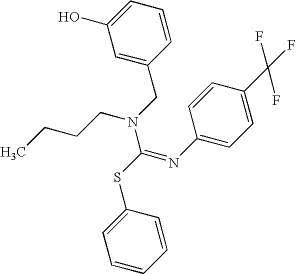 | C₂₅H₂₅F₃N₂OS | 458.546 |
| 10700 | 854 | 1 | 10700-011 | F 08 | 0.215 | 55.75296108 | Spy4H | 0.1776 | TR1070000854 | 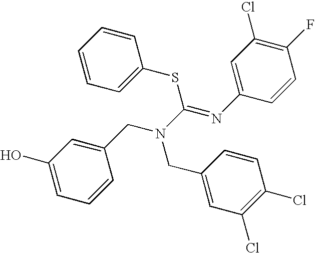 | C₂₇H₂₀Cl₃FN₂OS | 545.891 |
| 10700 | 124 | 1 | 10700-002 | D 07 | 0.215 | 55.62322371 | Spy4H | 0.1776 | TR1070000124 | 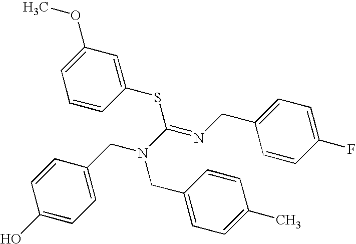 | C₃₀H₂₉FN₂O₂S | 500.635 |
| 10700 | 493 | 1 | 10700-007 | E 03 | 0.194 | 55.60165975 | Spy4H | 0.1776 | TR1070000493 | 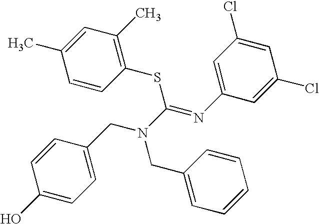 | C₂₉H₂₆Cl₂N₂OS | 521.509 |
| 10700 | 1076 | 1 | 10700-014 | D 06 | 0.198 | 55.09160631 | Spy4H | 0.1776 | TR1070001076 | 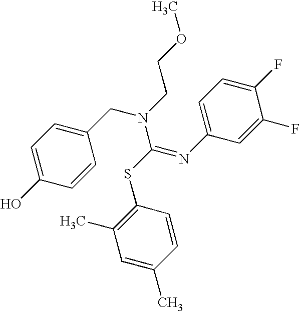 | C₂₅H₂₆F₂N₂O₂S | 456.554 |
| 10700 | 1044 | 1 | 10700-014 | D 02 | 0.184 | 55.09160631 | Spy4H | 0.1776 | TR1070001044 | 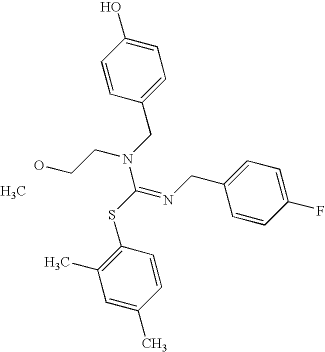 | C₂₆H₂₉FN₂O₂S | 452.591 |
| 10700 | 248 | 1 | 10700-004 | H 02 | 0.232 | 55.0405562 | Spy4H | 0.1776 | TR1070000248 | 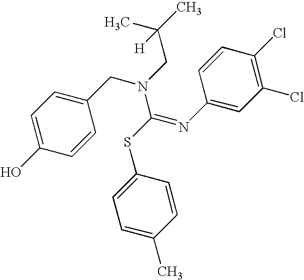 | C₂₅H₂₆Cl₂N₂OS | 473.465 |
| 10700 | 663 | 1 | 10700-009 | G 04 | 0.179 | 54.80018374 | Spy4H | 0.1776 | TR1070000663 | 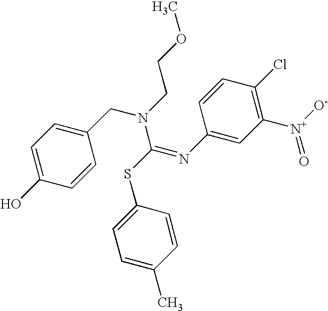 | C₂₄H₂₄ClN₃O₄S | 485.99 |
| 10700 | 1242 | 1 | 10700-016 | B 07 | 0.215 | 54.76244861 | Spy4H | 0.1776 | TR1070001242 | 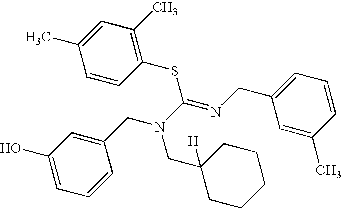 | C₃₁H₃₈N₂OS | 486.72 |
| 10700 | 522 | 1 | 10700-007 | B 07 | 0.288 | 54.60580913 | Spy4H | 0.1776 | TR1070000522 | 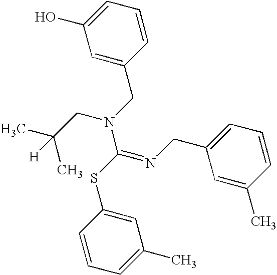 | C₂₇H₃₂N₂OS | 432.629 |
| 10700 | 913 | 1 | 10700-012 | A 06 | 0.181 | 54.57685665 | Spy4H | 0.1776 | TR1070000913 | 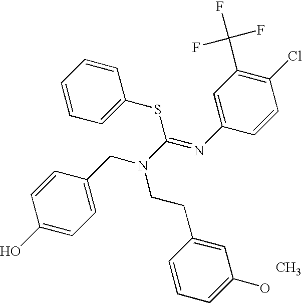 | C₃₀H₂₆ClF₃N₂O₂S | 571.06 |
| 10700 | 393 | 1 | 10700-005 | A 11 | 0.204 | 54.54202789 | Spy4H | 0.1776 | TR1070000393 | 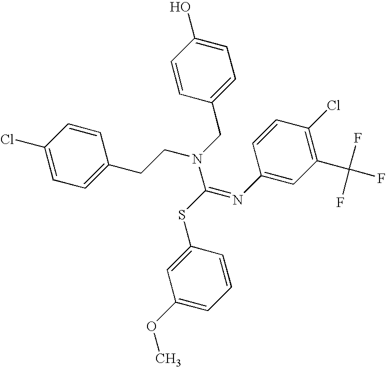 | C₃₀H₂₅Cl₂F₃N₂O₂S | 605.505 |
| 10700 | 783 | 1 | 10700-010 | G 09 | 0.187 | 54.52969224 | Spy4H | 0.1776 | TR1070000783 | 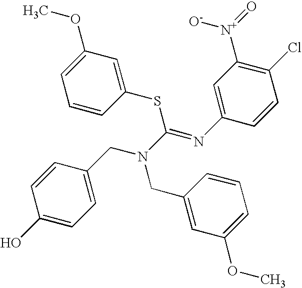 | C₂₉H₂₆ClN₃O₅S | 564.059 |
| 10700 | 1073 | 1 | 10700-014 | A 06 | 0.185 | 54.40988496 | Spy4H | 0.1776 | TR1070001073 | 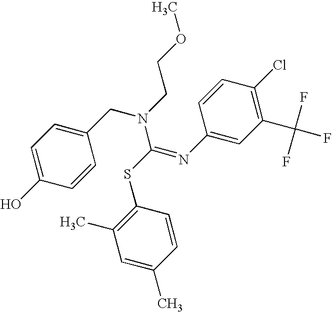 | C₂₆H₂₆ClF₃N₂O₂S | 523.016 |
| 10700 | 396 | 1 | 10700-005 | D 11 | 0.238 | 54.24048247 | Spy4H | 0.1776 | TR1070000396 | 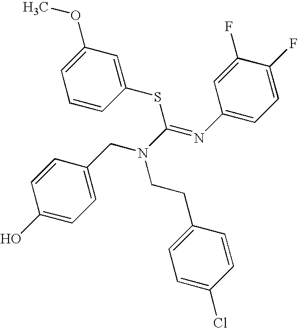 | C₂₉H₂₅ClF₂N₂O₂S | 539.044 |
| 10700 | 953 | 1 | 10700-012 | A 11 | 0.183 | 54.23143351 | Spy4H | 0.1776 | TR1070000953 | 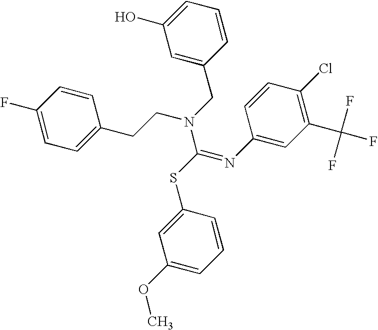 | C₃₀H₂₅ClF₄N₂O₂S | 589.05 |
| 10700 | 94 | 1 | 10700-002 | F 03 | 0.202 | 53.99918798 | Spy4H | 0.1776 | TR1070000094 | 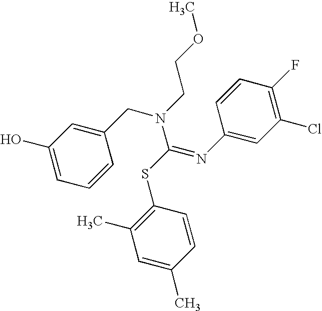 | C₂₅H₂₆ClFN₂O₂S | 473.009 |
| 10700 | 263 | 1 | 10700-004 | G 04 | 0.203 | 53.80455774 | Spy4H | 0.1776 | TR1070000263 | 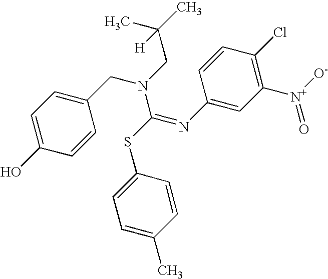 | C₂₅H₂₆ClN₃O₃S | 484.017 |
| 10700 | 1245 | 1 | 10700-016 | E 07 | 0.225 | 53.80310644 | Spy4H | 0.1776 | TR1070001245 | 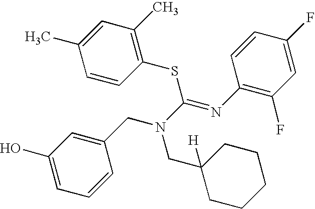 | C₂₉H₃₂F₂N₂OS | 494.647 |
| 10700 | 1244 | 1 | 10700-016 | D 07 | 0.232 | 53.80310644 | Spy4H | 0.1776 | TR1070001244 | 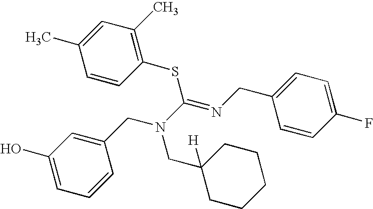 | C₃₀H₃₅FN₂OS | 490.684 |
| 10700 | 29 | 1 | 10700-001 | E 05 | 0.204 | 53.65010799 | Spy4H | 0.1776 | TR1070000029 | 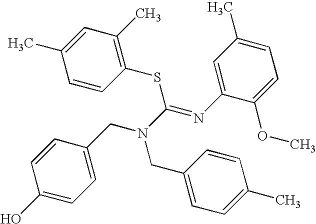 | C₃₂H₃₄N₂O₂S | 510.699 |
| 10700 | 1193 | 1 | 10700-015 | A 11 | 0.193 | 53.5729943 | Spy4H | 0.1776 | TR1070001193 | 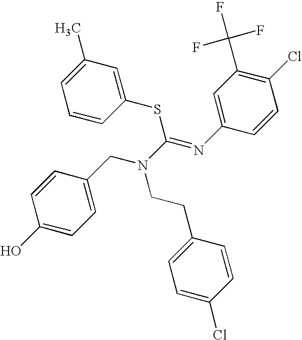 | C₃₀H₂₅Cl₂F₃N₂OS | 589.506 |
| 10700 | 1134 | 1 | 10700-015 | F 03 | 0.191 | 53.5729943 | Spy4H | 0.1776 | TR1070001134 | 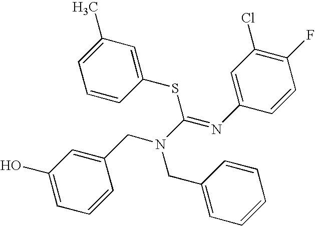 | C₂₈H₂₄ClFN₂OS | 491.028 |
| 10700 | 843 | 1 | 10700-011 | C 07 | 0.209 | 53.38409475 | Spy4H | 0.1776 | TR1070000843 | 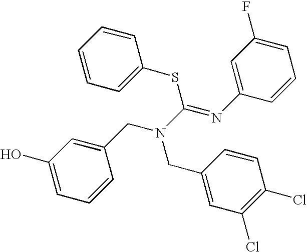 | C₂₇H₂₁Cl₂FN₂OS | 511.446 |
| 10700 | 1067 | 1 | 10700-014 | C 05 | 0.192 | 53.04644227 | Spy4H | 0.1776 | TR1070001067 | 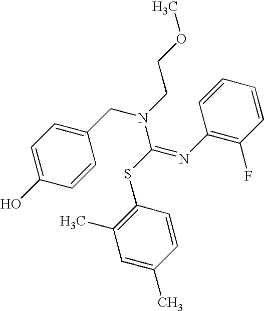 | C₂₅H₂₇FN₂O₂S | 438.564 |
| 10700 | 469 | 1 | 10700-006 | E 10 | 0.211 | 53.00613497 | Spy4H | 0.1776 | TR1070000469 | 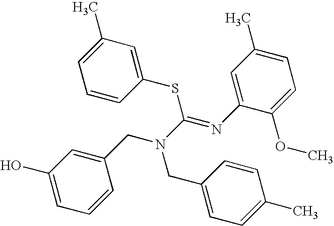 | C₃₁H₃₂N₂O₂S | 496.672 |
| 10700 | 658 | 1 | 10700-009 | B 04 | 0.254 | 52.96279283 | Spy4H | 0.1776 | TR1070000658 | 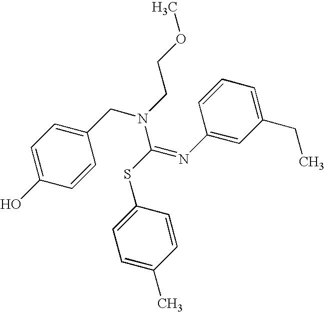 | C₂₆H₃₀N₂O₂S | 434.601 |
| 10700 | 513 | 1 | 10700-007 | A 06 | 0.197 | 52.94605809 | Spy4H | 0.1776 | TR1070000513 | 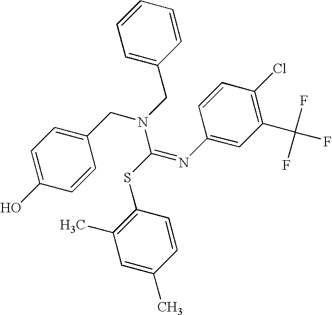 | C₃₀H₂₆ClF₃N₂OS | 555.061 |
| 10700 | 1186 | 1 | 10700-015 | B 10 | 0.195 | 52.87154757 | Spy4H | 0.1776 | TR1070001186 | 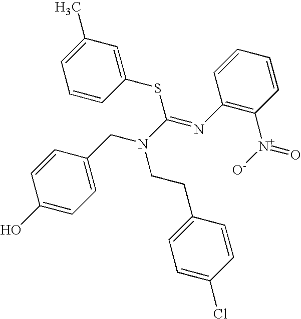 | C₂₉H₂₆ClN₃O₃S | 532.061 |
| 10700 | 343 | 1 | 10700-005 | G 04 | 0.225 | 52.73275537 | Spy4H | 0.1776 | TR1070000343 | 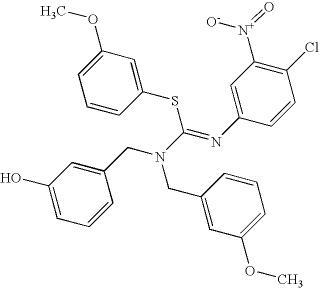 | C₂₉H₂₆ClN₃O₅S | 564.059 |
| 10700 | 52 | 1 | 10700-001 | D 08 | 0.195 | 52.61339093 | Spy4H | 0.1776 | TR1070000052 | 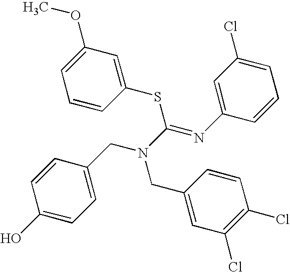 | C₂₈H₂₃Cl₃N₂O₂S | 557.927 |
| 10700 | 793 | 1 | 10700-010 | A 11 | 0.187 | 52.44906805 | Spy4H | 0.1776 | TR1070000793 | 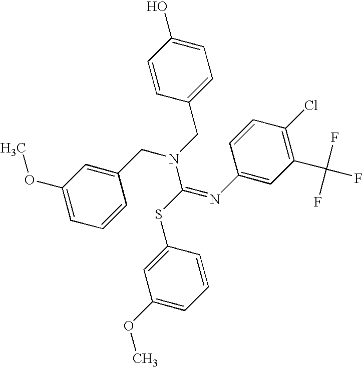 | C₃₀H₂₆ClF₃N₂O₃S | 587.059 |
| 10700 | 767 | 1 | 10700-010 | G 07 | 0.204 | 52.44906805 | Spy4H | 0.1776 | TR1070000767 | 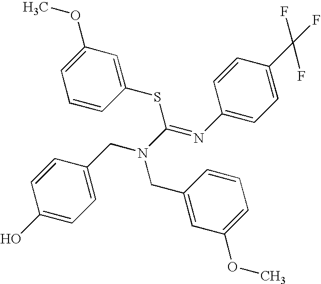 | C₃₀H₂₇F₃N₂O₃S | 552.614 |
| 10700 | 75 | 1 | 10700-001 | C 11 | 0.254 | 52.26781857 | Spy4H | 0.1776 | TR1070000075 | 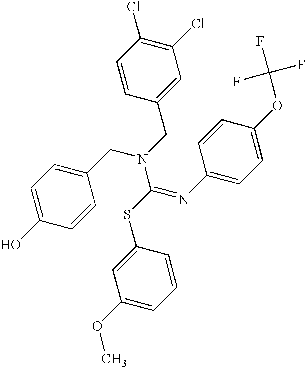 | C₂₉H₂₃Cl₂F₃N₂O₃S | 607.478 |
| 10700 | 1273 | 1 | 10700-016 | A 11 | 0.219 | 52.20420283 | Spy4H | 0.1776 | TR1070001273 | 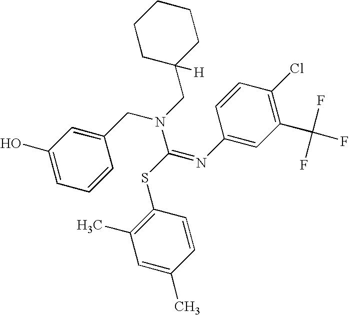 | C₃₀H₃₂ClF₃N₂OS | 561.109 |
| 10700 | 533 | 1 | 10700-007 | E 08 | 0.242 | 51.95020747 | Spy4H | 0.1776 | TR1070000533 | 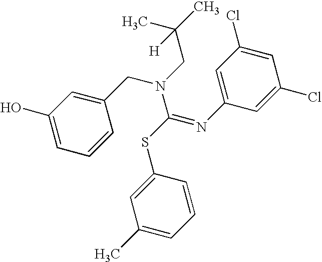 | C₂₅H₂₆Cl₂N₂OS | 473.465 |
| 10700 | 202 | 1 | 10700-003 | B 07 | 0.21 | 51.92159877 | Spy4H | 0.1776 | TR1070000202 | 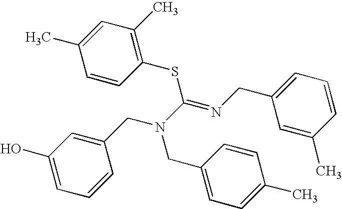 | C₃₂H₃₄N₂OS | 494.7 |
| 10700 | 1261 | 1 | 10700-016 | E 09 | 0.237 | 51.88442211 | Spy4H | 0.1776 | TR1070001261 | 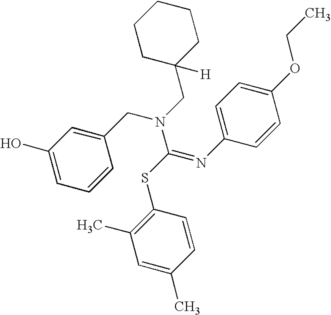 | C₃₁H₃₈N₂O₂S | 502.719 |
| 10700 | 433 | 1 | 10700-006 | A 06 | 0.203 | 51.69734151 | Spy4H | 0.1776 | TR1070000433 | 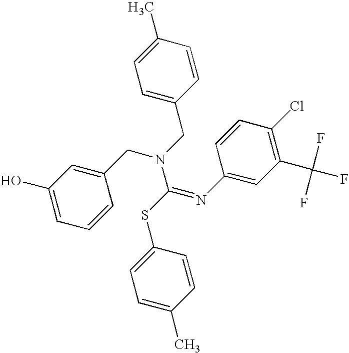 | C₃₀H₂₆ClF₃N₂OS | 555.061 |
| 10700 | 244 | 1 | 10700-004 | D 02 | 0.228 | 51.64156045 | Spy4H | 0.1776 | TR1070000244 | 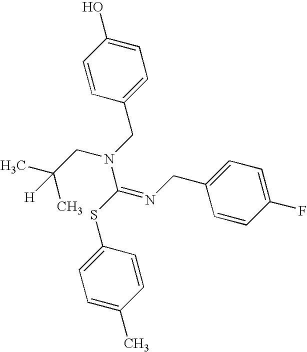 | C₂₆H₂₉FN₂OS | 436.592 |
| 10700 | 1128 | 1 | 10700-015 | H 02 | 0.207 | 51.4686541 | Spy4H | 0.1776 | TR1070001128 | 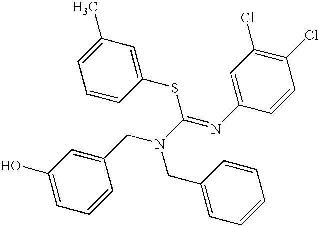 | C₂₈H₂₄Cl₂N₂OS | 507.483 |
| 10700 | 362 | 1 | 10700-005 | B 07 | 0.319 | 50.92348285 | Spy4H | 0.1776 | TR1070000362 | 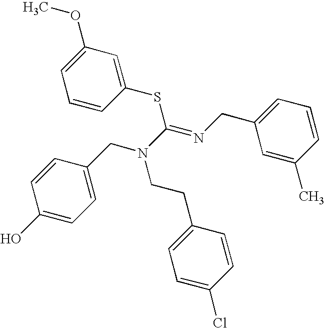 | C₃₁H₃₁ClN₂O₂S | 531.117 |
| 10700 | 943 | 1 | 10700-012 | G 09 | 0.218 | 50.77720207 | Spy4H | 0.1776 | TR1070000943 | 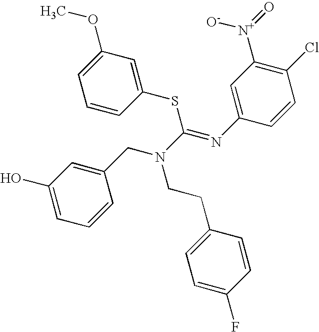 | C₂₉H₂₅ClFN₃O₄S | 566.05 |
| 10700 | 82 | 1 | 10700-002 | B 02 | 0.212 | 50.75111652 | Spy4H | 0.1776 | TR1070000082 | 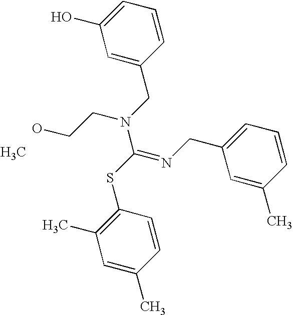 | C₂₇H₃₂N₂O₂S | 448.628 |
| 10700 | 521 | 1 | 10700-007 | A 07 | 0.202 | 50.62240664 | Spy4H | 0.1776 | TR1070000521 | 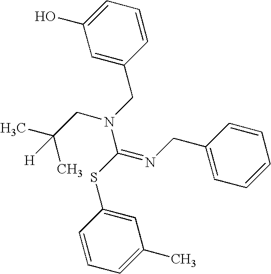 | C₂₆H₃₀N₂OS | 418.602 |
| 10700 | 334 | 1 | 10700-005 | F 03 | 0.225 | 50.62193743 | Spy4H | 0.1776 | TR1070000334 | 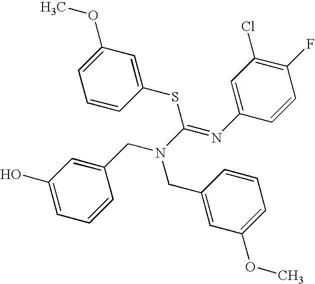 | C₂₉H₂₆ClFN₂O₃S | 537.052 |
| 10700 | 313 | 1 | 10700-004 | A 11 | 0.211 | 50.40556199 | Spy4H | 0.1776 | TR1070000313 | 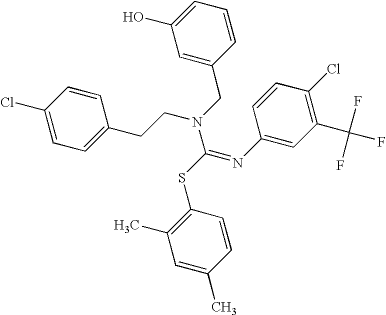 | C₃₁H₂₇Cl₂F₃N₂OS | 603.533 |
| 10700 | 363 | 1 | 10700-005 | C 07 | 0.285 | 50.32039201 | Spy4H | 0.1776 | TR1070000363 | 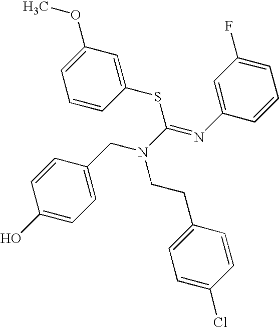 | C₂₉H₂₆ClFN₂O₂S | 521.053 |
| 10700 | 852 | 1 | 10700-011 | D 08 | 0.239 | 50 | Spy4H | 0.1776 | TR1070000852 | 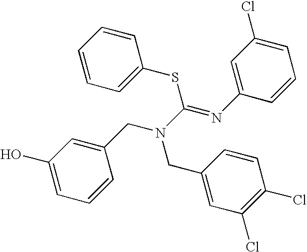 | C₂₇H₂₁Cl₃N₂OS | 527.901 |
| 10700 | 526 | 1 | 10700-007 | F 07 | 0.312 | 49.95850622 | Spy4H | 0.1776 | TR1070000526 | 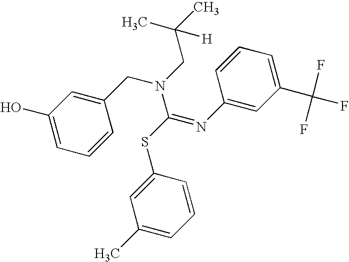 | C₂₆H₂₇F₃N₂OS | 472.572 |
| 10700 | 241 | 1 | 10700-004 | A 02 | 0.214 | 49.78756277 | Spy4H | 0.1776 | TR1070000241 | 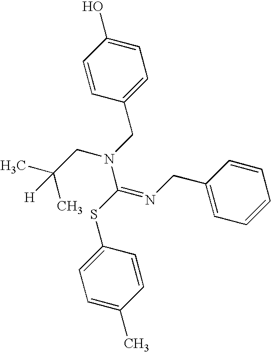 | C₂₆H₃₀N₂OS | 418.602 |
| 10700 | 282 | 1 | 10700-004 | B 07 | 0.218 | 49.78756277 | Spy4H | 0.1776 | TR1070000282 | 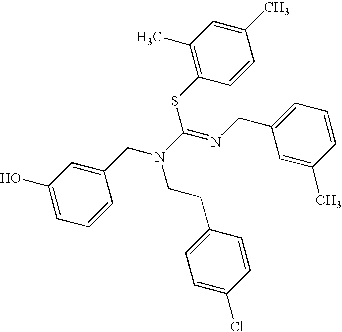 | C₃₂H₃₃ClN₂OS | 529.145 |
| 10700 | 709 | 1 | 10700-009 | E 10 | 0.206 | 49.65548921 | Spy4H | 0.1776 | TR1070000709 | 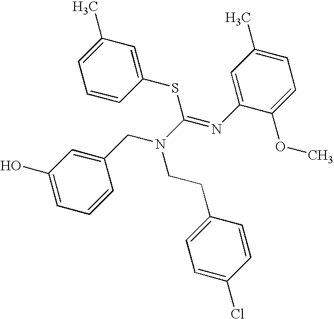 | C₃₁H₃₁ClN₂O₂S | 531.117 |
| 10700 | 168 | 1 | 10700-003 | H 02 | 0.222 | 49.46195234 | Spy4H | 0.1776 | TR1070000168 | 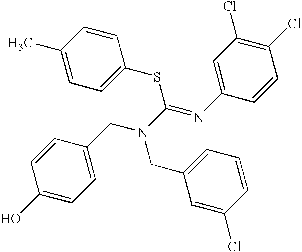 | C₂₈H₂₃Cl₃N₂OS | 541.928 |
| 10700 | 927 | 1 | 10700-012 | G 07 | 0.196 | 49.3955095 | Spy4H | 0.1776 | TR1070000927 | 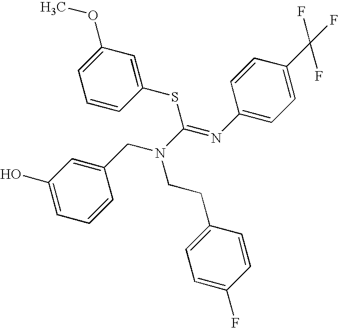 | C₃₀H₂₆F₄N₂O₂S | 554.605 |
| 10700 | 1155 | 1 | 10700-015 | C 06 | 0.207 | 49.3643139 | Spy4H | 0.1776 | TR1070001155 | 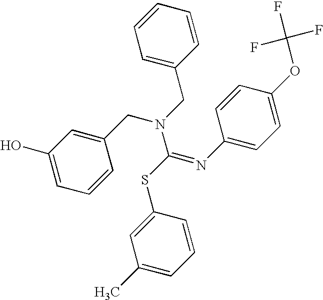 | C₂₉H₂₅F₃N₂O₂S | 522.589 |
| 10700 | 842 | 1 | 10700-011 | B 07 | 0.209 | 49.32318105 | Spy4H | 0.1776 | TR1070000842 | 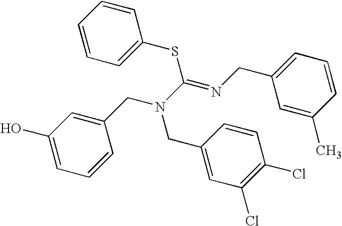 | C₂₉H₂₆Cl₂N₂OS | 521.509 |
| 10700 | 846 | 1 | 10700-011 | F 07 | 0.206 | 49.32318105 | Spy4H | 0.1776 | TR1070000846 | 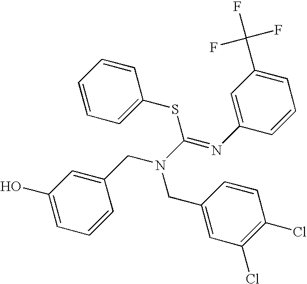 | C₂₈H₂₁Cl₂F₃N₂OS | 561.453 |
| 10700 | 254 | 1 | 10700-004 | F 03 | 0.231 | 49.16956354 | Spy4H | 0.1776 | TR1070000254 | 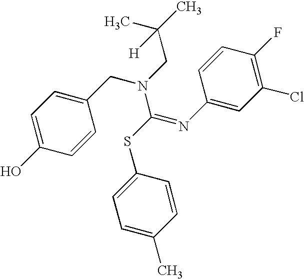 | C₂₅H₂₆ClFN₂OS | 457.01 |
| 10700 | 366 | 1 | 10700-005 | F 07 | 0.233 | 49.11421033 | Spy4H | 0.1776 | TR1070000366 | 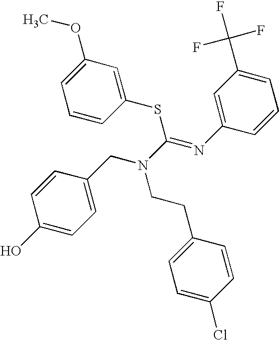 | C₃₀H₂₆ClF₃N₂O₂S | 571.06 |
| 10700 | 414 | 1 | 10700-006 | F 03 | 0.209 | 49.0797546 | Spy4H | 0.1776 | TR1070000414 | 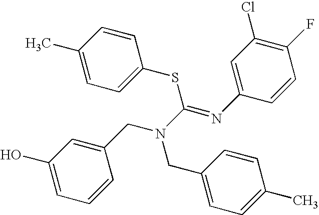 | C₂₉H₂₆ClFN₂OS | 505.054 MOLWEI |
| 10700 | 223 | 1 | 10700-003 | G 09 | 0.228 | 48.84704074 | Spy4H | 0.1776 | TR1070000223 | MOLSTRUCTURE | MOLFORMULA | GHT |
| | | | | | | | | | | 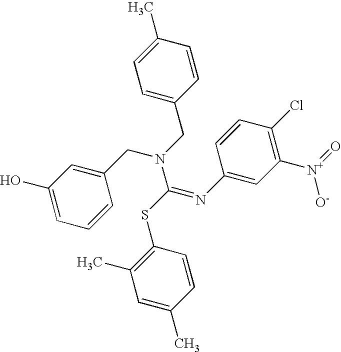 | | |

### EXAMPLE 3

### Melanocortin Receptor Assay

This example describes methods for assaying binding to MC receptors.

All cell culture media and reagents are obtained from GibcoBRL (Gaithersburg Md.), except for COSMIC CALF SERUM (HyClone; Logan Utah). HEK 293 cell lines are transfected with the human MC receptors hMCR-1, hMCR-3, and hMCR-4 (Gantz et al., Biochem. Biophys. Res. Comm. 200:1214-1220 (1994); Gantz et al., J. Biol. Chem. 268:8246-8250 (1993); Gantz et al. J. Biol. Chem. 268:15174-15179 (1993); Haskell-Leuvano et al., Biochem. Biophys. Res. Comm. 204:1137-1142 (1994); each of which is incorporated herein by reference). Vectors for construction of an hMCR-5 expressing cell line are obtained, and a line of HEK 293 cells expressing hMCR-5 is constructed (Gantz, supra, 1994). hMCR-5 has been described previously (Franberg et al., Biochem. Biophys. Res. Commun. 236:489-492 (1997); Chowdhary et al., Cytogenet. Cell Genet. 68:1-2 (1995); Chowdhary et al., Cytogenet. Cell Genet. 68:79-81 (1995), each of which is incorporated herein by reference). HEK 293 cells are maintained in DMEM, 25 mM HEPES, 2 mM glutamine, non-essential amino acids, vitamins, sodium pyruvate, 10% COSMIC CALF SERUM, 100 units/ml penicillin, 100 μg/ml streptomycin and 0.2 mg/ml G418 to maintain selection.

Before assaying, cells are washed once with phosphate buffered saline (“PBS”; without Ca2+ and Mg2+), and stripped from the flasks using 0.25% trypsin and 0.5 mM EDTA. Cells are suspended in PBS, 10% COSMIC CALF SERUM and 1 mM CaCl2. Cell suspensions are prepared at a density of 2×104 cells/ml for HEK 293 cells expressing hMCR-3, hMCR-4 or hMCR-5, and 1×105 cells/ml for HEK 293 cells expressing hMCR-1. Suspensions are placed in a water bath and allowed to warm to 37° C. for 1 hr.

Binding assays are performed in a total volume of 250 μl for HEK 293 cells. Control and test compounds are dissolved in distilled water. 125I-HP 467 (50,000 dpm) (2000 Ci/mmol) (custom labeled by Amersham; Arlington Heights Ill.) is prepared in 50 mM Tris, pH 7.4, 2 mg/ml BSA, 10 mM CaCl2, 5 mM MgCl2, 2 mM EDTA and added to each tube. To each tube is added 4×103 HEK 293 cells expressing hMCR-3, hMCR-4 or hMCR-5, or 2×104 cells expressing hMCR-1. Assays are incubated for 2.5 hr at 37° C.

GF/B filter plates are prepared by soaking for at least one hour in 5 mg/ml BSA and 10 mM CaCl2. Assays are filtered using a Brandel 96-well cell harvester (Brandel Inc.; Gaithersburg, Md.). The filters are washed four times with cold 50 mM Tris, pH 7.4, and the filter plates dehydrated for 2 hr and 35 μl of MICROSCINT is added to each well. Filter plates are counted using a Packard Topcount (Packard Instrument Co.) and data analyzed using GraphPad PRISM v2.0 (GraphPad Software Inc.; San Diego Calif.) and Microsoft EXCEL v5.0a (Microsoft Corp.; Redmond Wash.).

To assay S-aryl-isothiourea derivative compounds, binding assays are performed in duplicate in a 96 well format. HP 467 is prepared in 50 mM Tris, pH 7.4, and 125I-HP 467 is diluted to give 100,000 dpm per 50 μl. A S-aryl-isothiourea derivative compound, is added to the well in 25 μl aliquots. A 25 μl aliquot of 125I-HP 467 is added to each well. A 0.2 ml aliquot of suspended cells is added to each well to give the cell numbers indicate above, and the cells are incubated at 37° C. for 2.5 hr. Cells are harvested on GF/B filter plates as described above and counted.

### EXAMPLE 4

### Penile Erection Due to Administration of a S-aryl-isothiourea Derivative Compounds

Adult male rats are housed 2-3 per cage and are acclimated to the standard vivarium light cycle (12 hr. light, 12 hr. dark), rat chow and water for a least a week prior to testing. All experiments are performed between 9 a.m. and noon and rats are placed in cylindrical, clear plexiglass chambers during the 60 minute observation period. Mirrors are positioned below and to the sides of the chambers, to improve viewing.

Observations begin 10 minutes after an intraperitoneal injection of either saline or compound. An observer counts the number of grooming motions, stretches, yawns and penile erections (spontaneously occurring, not elicited by genital grooming) and records them every 5 minutes, for a total of 60 minutes. The observer is unaware of the treatment and animals are tested once, with n=6 in each group. Values in the figures represent the group mean and standard error of the mean. HP 228 can be used as a positive control for penile erections. Significant differences between groups are determined by an overall analysis of variance and the Student Neunmann-Keuls post hoc test can be used to identify individual differences between groups (p £0.05).

Although the invention has been described with reference to the examples provided above, it should be understood that various modifications can be made by those skilled in the art without departing from the invention. Accordingly, the invention is set out in the following claims.

We claim:

1. A combinatorial library of two or more compounds of the formula: 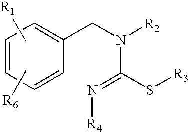
wherein
R1 and R6 are independently selected from the group consisting of H, —OH, halide, C₁ to C₇ alkoxy, substituted C₁ to C₇ alkoxy, amino, substituted amino, acylamino, substituted acylamino, carboxyl, carboxylate ester, carboxamide, substituted carboxamide, phenyl, substituted phenyl, C₁ to C₆ alkyl, C₁ to C₆ substituted alkyl, C₁ to C₆ alkenyl, C₁ to C₆ substituted alkenyl, C₁ to C₆ alkynyl, C₁ to C₆ substituted alkynyl, nitro, thiol, thioether, and substituted thioether, or R1 and R6 taken together form a ring fused to the phenyl to form a naphthyl ring,
R2 is selected from the group consisting of C₁ to C₆ alkyl, C₁ to C₆ substituted alkyl, benzyl, substituted benzyl, phenylalkyl and substituted phenylalkyl,
R3 is selected from the group consisting of phenyl, substituted phenyl naphthyl and substituted naphthyl; and
R4 is selected from the group consisting of H, and an unsubstituted or a substituted phenyl, wherein said substituted phenyl is substituted with at least one substituent selected from the group consisting of: halo, C₁ to C₆ alkyl, C₁ to C₆ alkoxy, nitro, C₁ to C₆ substituted alkyl, C₁ to C₆ substituted alkoxy, C₁ to C₆ alkylthio, phenyl, cyano, benzyl, substituted benzyl, naphthyl, and substituted naphthyl.

2. The combinatorial library according to claim 1, wherein said C₁ to C₆ substituted alkyl is substituted with at least one substituent selected from the group consisting of thiol, halo, C₁ to C₆ alkoxy, and phenyl unsubstituted or substituted with a substituent selected from the group consisting of halo and C₁ to C₆ alkoxy.

3. The combinatorial library according to claim 1, wherein said C₁ to C₆ substituted alkoxy is substituted with at least one substituent selected from the group consisting of thiol and halo.

4. The combinatorial library according to claim 1, wherein R₁ is —OH.

5. The combinatorial library according to claim 1, wherein R₂ is selected from the group consisting of 4-methylbenzyl, 3,4-dichlorobenzyl, 2-methoxyethyl, 3-chlorobenzyl, isobutyl, 2-(4-chlorophenyl)ethyl, 3-methoxybenzyl, phenyl, butyl,2-(3-methoxyphenyl)ethyl, 2-(4-florophenyl)ethyl, 4-methoxybenzyl, 2-ethoxyethyl, methylcyclohexyl, cyclohexyl, 3,4-dimethoxybenzyl,2-(4-methoxyphenyl)ethyl, and DL-alpha-methylbenzyl.

6. The combinatorial library according to claim 1, wherein R3 is selected from the group consisting phenyl, 2,4-dimethylphenyl, 3-methoxyphenyl, 3-methylphenyl, and 4-methylphenyl.

7. The combinatorial library according to claim 1, wherein R4 is selected from the group consisting of 2,4-difluorophenyl, 2-fluorophenyl, 2-methoxy-5-methylphenyl, 2-nitrophenyl, 3-(methylthio)phenyl, 3-(trifluoromethyl)phenyl, 3,4-dichlorophenyl, 3,4-difluorophenyl, 3,4-dimethylphenyl, 3,5-dichlorophenyl, 3,5-dimethoxyphenyl, 3,5-dimethylphenyl, 3-chloro-4-fluorophenyl, 3-chloro-4-methylphenyl, 3-chlorophenyl, 3-ethylphenyl, 3-fluoro-4-methylphenyl, 3-fluorophenyl, 3-methoxyphenyl, 3-methylbenzyl, 3-nitrophenyl, 4-(methylthio)phenyl, 4-(trifluoromethoxy)phenyl, 4-(trifluoromethyl)phenyl, 4-biphenylyl, 4-bromophenyl, 4-butoxyphenyl, 4-chloro-3-(trifluoromethyl)phenyl, 4-chloro-3-nitrophenyl, 4-chlorophenyl, 4-cyanophenyl, 4-ethoxyphenyl, 4-ethylphenyl, 4-fluoro-3-nitrophenyl, 4-fluorobenzyl, 4-isopropylphenyl, 4-methoxyphenyl, 4-n-butylphenyl, benzyl, and p-tolyl.

8. A compound of the formula: 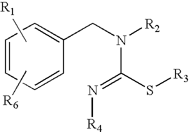
wherein
R1 and R6 are independently selected from the group consisting of H, —OH, halide, C₁ to C₇ alkoxy, substituted C₁ to C₇ alkoxy, amino, substituted amino, acylamino, substituted acylamino, carboxyl, carboxylate ester, carboxamide, substituted carboxamide, phenyl, substituted phenyl, C₁ to C₆ alkyl, C₁ to C₆ substituted alkyl, C₁ to C₆ alkenyl, C₁ to C₆ substituted alkenyl, C₁ to C₆ alkynyl, C₁ to C₆ substituted alkynyl, nitro, thiol, thioether, and substituted thioether,or R1 and R6 taken together form a ring fused to the phenyl to form a naphthyl ring,
R2 is selected from the group consisting of C₁ to C₆ alkyl, C₁ to C₆ substituted alkyl, benzyl, substituted benzyl, phenylalkyl and substituted phenylalkyl,
R3 is selected from the group consisting of phenyl, substituted phenyl, naphthyl and substituted naphthyl; and
R4 is selected from the group consisting of H, and an unsubstituted or a substituted phenyl, wherein said substituted phenyl is substituted with at least one substituent selected from the group consisting of: halo, C₁ to C₆ alkyl, C₁ to C₆ alkoxy, nitro, C₁ to C₆ substituted alkyl, C₁ to C₆ substituted alkoxy, C₁ to C₆ alkylthio, phenyl, cyano, benzyl, substituted benzyl, naphthyl, and substituted naphthyl.

9. The compound according to claim 8, wherein said C₁ to C₆ substituted alkyl is substituted with at least one substituent selected from the group consisting of thiol, halo, C₁ to C₆ alkoxy, and phenyl unsubstituted or substituted with a substituent selected from the group consisting of halo and C₁ to C₆ alkoxy.

10. The compound according to claim 8, wherein said C₁ to C₆ substituted alkoxy is substituted with at least one substituent selected from the group consisting of thiol and halo.

11. The compound according to claim 8, wherein R₁ is —OH.

12. The compound according to claim 8, wherein R₂ is selected from the group consisting of 4-methylbenzyl, 3,4-dichlorobenzyl, 2-methoxyethyl, 3-chlorobenzyl, isobutyl, 2-(4-chlorophenyl)ethyl, 3-methoxybenzyl, phenyl, butyl,2-(3-methoxyphenyl)ethyl, 2-(4-florophenyl)ethyl, 4-methoxybenzyl, 2-ethoxyethyl, methylcyclohexyl, cyclohexyl, 3,4-dimethoxybenzyl,2-(4-methoxyphenyl)ethyl, and DL-alpha-methylbenzyl.

13. The compound according to claim 8, wherein R₃ is selected from the group consisting of H, methyl, and methoxy.

14. The compound according to claim 8, wherein R₄ is selected from the group consisting of an unsubstituted or a substituted phenyl, wherein said substituted phenyl is substituted with at least one substituent selected from the group consisting of halo, methoxy, methyl, nitro, ethyl, methylthio, phenyl, butoxy, cyano, ethoxy, ethyl, and isopropyl.

15. A method of making the compound of claim 8, comprising
preparing a resin bound linker,
reacting said resin bound linker with an aldehyde substituted compound to produce a resin bound aldehyde compound,
reacting said resin bound aldehyde with an amine to produce a resin bound amine,
reacting said resin bound amine with di(benzotriazole-1-yl)methanimine to produce a resin bound benzotriazole amidine,
reacting said resin bound benzotriazole amidine with a thiophenol to produce a resin bound N,N-substituted N′ unsubstituted S-Aryl isothiourea, and
cleaving and extracting said N,N-substituted N′ unsubstituted S-Aryl isothiourea from said resin or reacting said resin bound N,N-substituted N′ unsubstituted S-Aryl isothiourea with an isocyanate to produce an N,N,N′ substituted S-Aryl isothiourea resin, and then cleaving and extracting said N,N,N′ substituted S-Aryl isothiourea from said resin.

16. The method according to claim 15, wherein said aldehyde is selected from the group consisting of 3-hydroxybenzaldehyde and 4-hydroxybenzaldehyde.

17. The method according to claim 15, wherein said linker is a Wang linker.

18. The method according to claim 15, wherein said resin is polystyrene resin.
